(19) United States
(12) Patent Application Publication
(10) Pub. No.: US 2013/0123124 A1
(43) Pub. Date: May 16, 2013

(54) METHODS AND COMPOSITIONS FOR CHARACTERIZING AUTISM SPECTRUM DISORDER BASED ON GENE EXPRESSION PATTERNS

(71) Applicants: Louis M. Kunkel, Westwood, MA (US); Isaac S. Kohane, Newton, MA (US); Sek Won Kong, Lexington, MA (US); Christin D. Collins, Brookline, MA (US)

(72) Inventors: Louis M. Kunkel, Westwood, MA (US); Isaac S. Kohane, Newton, MA (US); Sek Won Kong, Lexington, MA (US); Christin D. Collins, Brookline, MA (US)

(73) Assignee: Children's Medical Center Corporation, Boston, MA (US)

(21) Appl. No.: 13/634,300

(22) PCT Filed: Mar. 11, 2011
(86) PCT No.: PCT/US11/28142

§ 371 Date: Jan. 24, 2013

Related U.S. Application Data
(60) Provisional application No. 61/313,565, filed on Mar. 12, 2010.

(51) Int. Cl. C12Q 1/68

(52) U.S. Cl. CPC C12Q 1/6813
USPC 506/9; 506/16

(57) ABSTRACT

The invention relates to methods and kits for characterizing and diagnosing autism spectrum disorder in an individual based on gene expression levels.

### RELATED APPLICATIONS

This application claims priority under 35 U.S.C. §119 from U.S. provisional application Ser. No. 61/313,565, filed Mar. 12, 2010. The entire teachings of the referenced provisional application is expressly incorporated herein by reference.

### FUNDING

This invention was made with United States Government support under grants R01 MH085143 and P30HD018655 awarded by the National Institutes of Health. The United States government has certain rights in the invention.

### FIELD OF THE INVENTION

The invention relates to methods and reagents for characterizing and diagnosing autism spectrum disorder.

### BACKGROUND OF INVENTION

Autism Spectrum Disorders (ASD) cover a broad spectrum of neurocognitive and social developmental delays with typical onset before 3 years of age including Autistic Disorder, Pervasive Developmental Disorder-Not Otherwise Specified and Asperger's Disorder as sub classified in the Diagnostic and Statistical Manual of Psychiatric Disorders, 4th edition, Text Revision (DSM-IV-TR). Prevalence of ASD has been increasing during last decades, and current estimation is 1 in 90 (Kogan, M. D., et al. Prevalence of parent-reported diagnosis of autism spectrum disorder among children in the US, 2007. Pediatrics 124, 1395-1403 (2009)) to 3.7 in 1000². There are waiting lists for evaluation by most centers with expertise, and despite the progress made in adopting instruments such as the Autism Diagnostic Interview-Revised (ADI-R) and the Autism Diagnostic Observation Schedule (ADOS) there remains significant debate regarding the prognostic value and accuracy of existing instruments³. Thus, improved diagnostic approaches are needed.

### SUMMARY OF THE INVENTION

It has been discovered that a variety of genes are differentially expressed in individuals having autism spectrum disorder compared with individuals free of autism spectrum disorder. Such genes are identified as autism spectrum disorder-associated genes. It has also been discovered that the autism spectrum disorder status of an individual can be classified with a high degree of accuracy, sensitivity, and specificity based on expression levels of these autism spectrum disorder-associated genes. Accordingly, methods and related kits are provided herein for characterizing and diagnosing autism spectrum disorder in an individual.

According to some aspects of the invention, methods of characterizing the autism spectrum disorder status of an individual in need thereof are provided. In some embodiments, the methods comprise: (a) obtaining a clinical sample from the individual; (b) determining expression levels of a plurality of autism spectrum disorder-associated genes in the clinical sample using an expression level determining system, wherein the autism spectrum disorder-associated genes comprise at least ten genes selected from Table 4, 5, 6, 7 or 10; and (c) comparing each expression level determined in (b) with an appropriate reference level, wherein the results of comparing in (c) characterize the autism spectrum disorder status of the individual. In some embodiments, the methods further comprise diagnosing autism spectrum disorder in the individual based on the autism spectrum disorder status. In some embodiments, the autism spectrum disorder-associated genes comprise at least one of: ARRB2, AVIL, BTBD14A, CCDC50, CD180, CD300LF, CPNE5, CXCL1, CYP4F3, FAM101B, FAM13A1OS, HAL, KCNE3, LOC643072, LTB4R, MAN2A2, MSL-1, MYBL2, NBEAL2, NFAM1, NHS, PLA2G7, PNOC, RASSF6, REM2, SIRPA, SLC45A4, SPIB, SULF2, TMEM190, ZNF516, and ZNF746. In some embodiments, a higher level of at least one autism spectrum disorder-associated gene selected from: ARRB2, AVIL, BTBD14A, CD300LF, CXCL1, CYP4F3, FAM101B, FAM13A1OS, HAL, KCNE3, LOC643072, LTB4R, MAN2A2, MSL-1, NBEAL2, NFAM1, NHS, PLA2G7, REM2, SIRPA, SLC45A4, SULF2, and ZNF746, compared with an appropriate reference level, characterizes the individual's autism spectrum disorder status as having autism spectrum disorder. In some embodiments, a lower level of at least one autism spectrum disorder-associated gene selected from: CCDC50, CD180, CPNE5, MYBL2, PNOC, RASSF6, and SPIB, compared with an appropriate reference level, characterizes the individual's autism spectrum disorder status as having autism spectrum disorder. In some embodiments, the autism spectrum disorder-associated genes comprise at least one of: BCL11A, BLK, C5orf13, CCDC50, CD180, CENPM, CPNE5, CTBP2, EBF1, EIF1AY, FAM105A, FCRL2, HEBP2, IGL@, LOC401233, LRRC6, PLA2G7, PMEPA1, PNN, PNOC, POU2AF1, PRICKLE1, RBP7, SPIB, SULF2, TCF4, TUBB2A, ZNF117, ZNF20, ZNF763, and ZNF830. In some embodiments, the autism spectrum disorder-associated genes comprise at least one of: TSNAX, SH3BP5L, PPIF, CCDC6, CTSD, IL18, UFM1, MTRF1, LPAR6, TWSG1, MAPKSP1, CD180, NFYA, TTRAP, ZNF92, CAPZA2, BLK, OSTF1, HSDL2, ATP6V1G1, DCAF12, and NOTCH1. In some embodiments, the clinical sample is a sample of peripheral blood, brain tissue, or spinal fluid. In some embodiments, each expression level is a level of an RNA encoded by an autism spectrum disorder-associated gene of the plurality. In some embodiments, the expression level determining system comprises a hybridization-based assay for determining the level of the RNA in the clinical sample. In some embodiments, the hybridization-based assay is an oligonucleotide array assay, an oligonucleotide conjugated bead assay, a molecular inversion probe assay, a serial analysis of gene expression (SAGE) assay, or an RT-PCR assay. In some embodiments, each expression level is a level of a protein encoded by an autism spectrum disorder-associated gene of the plurality. In some embodiments, the expression level determining system comprises an antibody-based assay for determining the level of the protein in the clinical sample. In some embodiments, the antibody-based assay is an antibody array assay, an antibody conjugated-bead assay, an enzyme-linked immuno-sorbent (ELISA) assay, or an immunoblot assay.

According to some aspects of the invention, the methods of characterizing the autism spectrum disorder status in an individual in need thereof comprise (a) obtaining a peripheral blood sample from the individual; (b) determining expression levels of a plurality of autism spectrum disorder-associated genes in the clinical sample using an expression level determining system, wherein the autism spectrum disorder-associated genes comprise at least ten genes selected from Table 4, 5, 6, 7 or 10; and (c) applying an autism spectrum disorder-classifier to the expression levels, wherein the autism spectrum disorder-classifier characterizes the autism spectrum disorder status of the individual based on the expression levels. In some embodiments, the methods further comprise diagnosing autism spectrum disorder in the individual based on the autism spectrum disorder status. In some embodiments, the autism spectrum disorder-classifier comprises an algorithm selected from logistic regression, partial least squares, linear discriminant analysis, quadratic discriminant analysis, neural network, naïve Bayes, C4.5 decision tree, k-nearest neighbor, random forest, and support vector machine. In some embodiments, the autism spectrum disorder-classifier has an accuracy of at least 75%. In some embodiments, the autism spectrum disorder-classifier has an accuracy in a range of about 75% to 90%. In some embodiments, the autism spectrum disorder-classifier has a sensitivity of at least 70%. In some embodiments, the autism spectrum disorder-classifier has a sensitivity in a range of about 70% to about 95%.

In some embodiments, the autism spectrum disorder-classifier has a specificity of at least 65%. In some embodiments, the autism spectrum disorder-classifier has a specificity in range of about 65% to about 85%. In some embodiments, the autism spectrum disorder-classifier is trained on a data set comprising expression levels of the plurality of autism spectrum disorder-associated genes in clinical samples obtained from a plurality of individuals identified as having autism spectrum disorder, wherein the interquartile range of ages of the plurality of individuals identified as having autism spectrum disorder is from about 2 years to about 10 years. In some embodiments, the autism spectrum disorder-classifier is trained on a data set comprising expression levels of the plurality of autism spectrum disorder-associated genes in clinical samples obtained from a plurality of individuals identified as not having autism spectrum disorder, wherein the interquartile range of ages of the plurality of individuals identified as not having autism spectrum disorder is from about 2 years to about 10 years. In some embodiments, the autism spectrum disorder-classifier is trained on a data set consisting of expression levels of the plurality of autism spectrum disorder-associated genes in clinical samples obtained from a plurality of male individuals. In some embodiments, the autism spectrum disorder-classifier is trained on a data set comprising expression levels of the plurality of autism spectrum disorder-associated genes in clinical samples obtained from a plurality of individuals identified as having autism spectrum disorder based on DSM-IV-TR criteria. In some embodiments, the autism spectrum disorder-associated genes comprise at least one of: BCL11A, BLK, C5orf13, CCDC50, CD180, CENPM, CPNE5, CTBP2, EBF1, EIF1AY, FAM105A, FCRL2, HEBP2, IGL@, LOC401233, LRRC6, PLA2G7, PMEPA1, PNN, PNOC, POU2AF1, PRICKLE1, RBP7, SPIB, SULF2, TCF4, TUBB2A, ZNF117, ZNF20, ZNF763, and ZNF830. In some embodiments, the autism spectrum disorder-associated genes comprise: TSNAX, SH3BP5L, PPIF, CCDC6, CTSD, IL18, UFM1, MTRF1, LPAR6, TWSG1, MAPKSP1, CD180, NFYA, TTRAP, ZNF92, CAPZA2, BLK, OSTF1, HSDL2, ATP6V1G1, DCAF12, and NOTCH1. In some embodiments, the autism spectrum disorder-associated genes comprise at least one of: ARRB2, AVIL, BTBD14A, CCDC50, CD180, CD300LF, CPNE5, CXCL1, CYP4F3, FAM101B, FAM13A1OS, HAL, KCNE3, LOC643072, LTB4R, MAN2A2, MSL-1, MYBL2, NBEAL2, NFAM1, NHS, PLA2G7, PNOC, RASSF6, REM2, SIRPA, SLC45A4, SPIB, SULF2, TMEM190, ZNF516, and ZNF746. In some embodiments, the clinical sample is a sample of peripheral blood, brain tissue, or spinal fluid. In some embodiments, each expression level is a level of an RNA encoded by an autism spectrum disorder-associated gene of the plurality. In some embodiments, the expression level determining system comprises a hybridization-based assay for determining the level of the RNA in the clinical sample. In some embodiments, the hybridization-based assay is an oligonucleotide array assay, an oligonucleotide conjugated bead assay, a molecular inversion probe assay, a serial analysis of gene expression (SAGE) assay, or an RT-PCR assay. In some embodiments, each expression level is a level of a protein encoded by an autism spectrum disorder-associated gene of the plurality. In some embodiments, the expression level determining system comprises an antibody-based assay for determining the level of the protein in the clinical sample. In some embodiments, the antibody-based assay is an antibody array assay, an antibody conjugated-bead assay, an enzyme-linked immuno-sorbent (ELISA) assay, or an immunoblot assay.

According to some aspects of the invention, arrays are provided that comprise, or consist essentially of, oligonucleotide probes that hybridize to nucleic acids having sequence correspondence to mRNAs of at least ten autism spectrum disorder-associated genes selected from Table 4, 5, 6, 7 or 10. According to other aspects of the invention, arrays are provided that comprise, or consist essentially of, antibodies that bind specifically to proteins encoded by at least ten autism spectrum disorder-associated genes selected from Table 4, 5, 6, 7 or 10.

According to some aspects of the invention, methods of monitoring progression of an autism spectrum disorder in an individual in need thereof are provided. In some embodiments, the methods comprise: (a) obtaining a clinical sample from the individual; (b) determining expression levels of a plurality of autism spectrum disorder-associated genes in the clinical sample using an expression level determining system, (c) comparing each expression level determined in (b) with an appropriate reference level, wherein the results of the comparison are indicative of the extent of progression of the autism spectrum disorder in the individual.

In some embodiments, the methods of monitoring progression of an autism spectrum disorder comprise: (a) obtaining a first clinical sample from the individual, (b) determining expression levels of a plurality of autism spectrum disorder-associated genes in the first clinical sample using an expression level determining system, (c) obtaining a second clinical sample from the individual, (d) determining expression levels of the plurality of autism spectrum disorder-associated genes in the second clinical sample using an expression level determining system, (e) comparing the expression level of each autism spectrum disorder-associated gene determined in (b) with the expression level determined in (d) of the same autism spectrum disorder associated-gene, wherein the results of comparing in (e) are indicative of the extent of progression of the autism spectrum disorder in the individual. In some embodiments, the autism spectrum disorder-associated genes comprise at least ten genes selected from Table 4, 5, 6, 7 or 10.

In some embodiments, the methods of monitoring progression of an autism spectrum disorder comprise: (a) obtaining a first clinical sample from the individual, (b) obtaining a second clinical sample from the individual, (c) determining the expression level of an autism spectrum disorder-associated gene in the first clinical sample using an expression level determining system, (d) determining the expression level of the autism spectrum disorder-associated gene in the second clinical sample using an expression level determining system, (e) comparing the expression level determined in (c) with the expression level determined in (d), and (f) repeating (c)-(e) for at least one other autism spectrum disorder-associated gene, wherein the results of comparing in (e) for the at least two autism spectrum-associated genes are indicative of the extent of progression of the autism spectrum disorder in the individual.

In some embodiments, the methods of monitoring progression of an autism spectrum disorder comprise: (a) obtaining a first clinical sample from the individual, (b) obtaining a second clinical sample from the individual, (c) determining a first expression pattern comprising expression levels of at least two autism spectrum disorder-associated genes in the first clinical sample using an expression level determining system, (d) determining a second expression pattern comprising expression levels of at least two autism spectrum disorder-associated genes in the second clinical sample using an expression level determining system, (e) comparing the first expression pattern with the second expression pattern, wherein the results of comparing in (e) are indicative of the extent of progression of the autism spectrum disorder in the individual.

In some embodiments of the methods of monitoring progression of an autism spectrum disorder, the time between obtaining the first clinical sample and obtaining the second clinical sample is a time sufficient for a change in the severity of the autism spectrum disorder to occur in the individual. In some embodiments, the individual is treated for the autism spectrum associated disorder between obtaining the first clinical sample and obtaining the second clinical sample.

According to some aspects of the invention, methods of assessing the efficacy of a treatment for an autism spectrum disorder in an individual in need thereof are provided. In some embodiments, the methods comprise: (a) obtaining a clinical sample from the individual, (b) administering a treatment to the individual for the autism spectrum disorder, (c) determining an expression pattern comprising expression levels of at least two autism spectrum disorder-associated genes in the clinical sample, (e) comparing the expression pattern with an appropriate reference expression pattern, wherein the appropriate reference expression pattern comprises expression levels of the at least two autism spectrum disorder-associated genes in a clinical sample obtained from a individual who does not have the autism spectrum disorder, wherein the results of the comparison in (c) are indicative of the efficacy of the treatment.

According to some aspects of the invention, the methods of assessing the efficacy of a treatment for an autism spectrum disorder comprise: (a) obtaining a first clinical sample from the individual, (b) administering a treatment to the individual for the autism spectrum disorder, (c) obtaining a second clinical sample from the individual after having administered the treatment to the individual, (d) determining a first expression pattern comprising expression levels of at least two autism spectrum disorder-associated genes in the first clinical sample, (e) comparing the first expression pattern with an appropriate reference expression pattern, wherein the appropriate reference expression pattern comprises expression levels of the at least two autism spectrum disorder-associated genes in a clinical sample obtained from a individual who does not have the autism spectrum disorder, (f) determining a second expression pattern comprising expression levels of at least two autism spectrum disorder-associated genes in the second clinical sample, and (g) comparing the second expression pattern with the appropriate reference expression pattern, wherein a difference between the second expression pattern and the appropriate reference expression pattern that is less than the difference between the first expression pattern and the appropriate reference pattern is indicative of the treatment being effective.

According to some aspects of the invention, methods for selecting an appropriate dosage of a treatment for an autism spectrum associated disorder in an individual in need thereof are provided. In some embodiments, the methods comprise: (i) administering a first dosage of a treatment for an autism spectrum associated disorder to the individual, (ii) assessing the efficacy of the first dosage of the treatment, in part, by determining at least one expression pattern comprising expression levels of at least two autism spectrum disorder-associated genes in a clinical sample obtained from the individual, (iii) administering a second dosage of a treatment for an autism spectrum associated disorder in the individual, (iv) assessing the efficacy of the second dosage of the treatment, in part, by determining at least one expression pattern comprising expression levels of at least two autism spectrum disorder-associated genes in a clinical sample obtained from the individual, wherein the appropriate dosage is selected as the dosage administered in (i) or (iii) that has the greatest efficacy. In some embodiments, the efficacy is assessed in (ii) and/or (iv) according to the methods disclosed herein.

According to some aspects of the invention, methods for selecting an appropriate dosage of a treatment for an autism spectrum associated disorder in an individual in need thereof are provided. In some embodiments, the methods comprise: (i) administering a dosage of a treatment for an autism spectrum associated disorder to the individual; (ii) assessing the efficacy of the dosage of the treatment, in part, by determining at least one expression pattern comprising expression levels of at least two autism spectrum disorder-associated genes in a clinical sample obtained from the individual, and (iii) selecting the dosage as being appropriate for the treatment for the autism spectrum associated disorder in the individual, if the efficacy determined in (ii) is at or above a threshold level, wherein the threshold level is an efficacy level at or above which a treatment substantially improves at least one symptom of an autism spectrum disorder.

According to some aspects of the invention, methods for identifying an agent useful for treating an autism spectrum associated disorder in an individual in need thereof are provided. In some embodiments, the methods comprise: (i) contacting an autism spectrum disorder-associated cell or tissue with a test agent, (ii) determining at least one expression pattern comprising expression levels of at least two autism spectrum disorder-associated genes in the autism spectrum disorder-associated cell or tissue, (iii) comparing the at least one expression pattern with a test expression pattern, and (iv) identifying the agent as being useful for treating the autism spectrum associated disorder based on the comparison in (iii). In some embodiments, the test expression pattern is an expression pattern indicative of an individual who does not have the autism spectrum disorder, and a decrease in a difference between the at least one expression pattern and the test expression pattern resulting from contacting the autism spectrum disorder-associated cell or tissue with the test agent identifies the test agent as being useful for the treatment of the autism spectrum associated disorder. In some embodiments, the autism spectrum disorder-associated cell or tissue is contacted with the test agent in (i) in vivo. In some embodiments, the autism spectrum associated disorder-cell or tissue is contacted with the test agent in (i) in vitro.

### BRIEF DESCRIPTION OF DRAWINGS

FIG. 1 shows an example study design and an overview of blood gene expression profiles. As show in FIG. 1a, 196 patients with ASDs and 182 controls are recruited for blood gene expression profiling. The blood samples from the first sample cohort (97 male patients with ASDs and 73 controls, P1) are prepared with Affymetrix HG-U133 Plus 2.0 arrays. P1 serves as a training set in building prediction models using blood gene expression changes. The second population dataset, P2, is prepared with Affymetrix Gene 1.0 ST array. The prediction models built with P1 were used to distinguish the ASD group from controls. The gene expression signature from P1 and P2 was compared to the postmortem brain samples that are hybridized to Affymetrix Exon 1.0 ST arrays. FIG. 1b shows global gene expression profile of P1 and P2 samples. After selecting the best matching probesets (see Example 6 for a description of methods) between two platforms, principal component analysis is performed to project samples in first two principal components. The difference between two datasets is minimal after normalization.

FIG. 2 shows prediction performance of ASD330 predictor genes on the training set (P1). As shown in FIG. 2a, Receiver Operating Characteristic (ROC) curve analysis is performed from logistic regression with 5-fold cross-validation. The area under ROC curve (AUC) is 0.88. ROC curve (gray line) is smoothed using a non-linear curve fitting (dark red line). Dotted blue line represents random classification accuracy (AUC=0.5). To estimate the robust prediction performance, 7 prediction algorithms were used; Partial Least Squares, Logistic Regression, C4.5 Decision Tree, Naïve Bayes, k-Nearest Neighbors, Random Forest, and Support Vector Machine (see Methods). Detailed prediction performances are summarized in Table 8. As shown in FIG. 2b, a biclustering method was used to cluster P1 samples (rows in the heatmap) and ASD330 genes (columns in the heatmap). The heatmap and dendrograms show the hierarchical biclustering of the normalized gene expression profiles for 170 samples in P1. Each row represents the normalized expression levels of a sample, and green (control) and orange (ASDs) colour bar shows the diagnostic phenotype of the sample. Consistent with a certain misclassification of samples in the predication analysis, not all ASD samples are clustered together.

FIG. 3 depicts prediction performances of ASD330 on the validation set (P2). As shown in FIG. 3a, 238 genes of ASD330 are best-matched in both P1 and P2 platform. When a prediction model is trained using the training set (P1), the prediction performance with the validation set (P2) measured by AUC is 0.76. As shown in FIG. 3b, AUC of the same predictors genes of P2 platform (ASD238) but retrained with 80% of P2 samples is 0.83 using a logistic regression. Gray line shows the actual data, and dark red line is smoothed after curve fitting.

FIG. 4 depicts blood gene expression signature discriminates postmortem brain gene expression profiles of ASD from control. FIG. 4a shows a comparison of differentially expressed genes (two group comparison p<0.01 for each dataset) between blood (P1 and P2) and brain datasets. 95 genes are in common between P1 and brain dataset, and 48 between P2 and brain dataset. As shown in FIG. 4b, a principal components analysis using ASD330 genes reveals a good separation between ASD and control samples (Hotelling's T² test permutation p-value 0.097). Two standard deviation ellipses are generated for ASD (red) and control (blue). Green arrowheads denote the outlier samples. FIG. 4c shows a prediction model using ASD330 genes identifies 9 out of 11 ASD samples correctly (AUC 0.95). All control samples are predicted as control, and two misclassified samples are the outlier samples in FIG. 4b (green arrowheads).

FIG. 5 shows significantly enriched gene sets for the prediction model, ASD330. FIG. 5a. shows those sets with p-values (uncorrected) less than 0.01 and the gene identities of the genes in those sets and also in the ASD330 are listed only if they number less than 50. Enriched genesets are categorized into 4 groups. FIG. 5b shows the distances of each sample from the overall centroid are calculated for 4 enriched categories list in FIG. 5a. The location of each sample represents relative enrichments of pathways. Blue and red ellipses denote the 1 standard deviation from the centroid. Most control samples are located close to zero, and ASD samples are more heterogeneously distributed. Some ASD samples with high immune response signature are not enriched for Synaptic plasticity (see FIG. 8 for all enriched pathways).

FIG. 6 depicts enriched Gene Ontology categories in both P1 and P2 datasets. A Cytoscape plug-in, ClueGO (available on the web at ici.upmc.fr/cluego/), was used to identify the Gene Ontology (GO) terms enriched in both datasets. The “detailed” biological process GO terms placed in GO levels 9-14 were primarily used. GO terms were grouped if a majority of genes were shared between two GO terms (Cohen's Kappa >0.5). The red circle represents the three GO terms related to the neuron differentiation, which was one of the common GO terms enriched between P1 and P2.

FIG. 7 shows region, age, and side enrichments of differentially expressed genes from postmortem cerebella samples. The human fetal brain 4 dataset (available at the Gene Expression Omnibus database with the accession ID GSE13344) was compared with the differentially expressed genes from our postmortem cerebella samples. The differential expression did not appear to be correlated to (FIG. 7a) a specific region, (FIG. 7b) age, or (FIG. 7c) side of the brain using onesided Wilcoxon rank sum tests. FIG. 7d shows multiple parts of brain at different ages were enriched. x-axis in each plot represented the −log(p-value), and samples were grouped as described in y-axis. The ages in FIG. 7b were weeks after gestation. In FIG. 7d, y-axis represented each brain sample with the sample naming scheme of [brain region]_[age(wks)][side].CEL. The brain region abbreviations are PFC: Prefrontal cortex, OPFC: Orbital PFC, DLPFC: dorsolateral PFC, MPFC medial PFC, VLPFC: ventrolateral PFC, HIP: hippocampus, STR: striatum, THM: thalamus, CBL: cerebellum, MS: motor-somatosensory cortex, Aud: auditory cortex, Occ: occipital cortex, and Par: parietal cortex.

FIG. 8 shows individual enrichments of gene sets. For enriched gene sets, multivariate distances of each sample from the centroid were calculated using Hotelling's T2 statistics. Each point in the scatterplot matrix represented a sample, and red and blue ellipses represented 2 standard deviations for ASDs and controls for given two gene sets. For any two gene sets, most samples were located within 2 standard deviations. The outliers that were mostly enriched with 6 a gene set were located off diagonal suggesting different subgroups were enriched with different gene sets.

FIG. 9 illustrates a prediction analysis. The prediction model selection procedure consisted of three nested loops. The outer most loop was the selection of top top N genes (10-1000) from ranked list by pAUC scores. The second loop was a leave-group out cross validation approach, where 80% of samples were randomly selected as a train set while maintaining the proportion of each diagnostic class. This step was repeated 500 times for each list of the top N genes. The inner most loop was used to optimize the parameters that were specific to a machine learning method used for a train set from an outer loop. This parameter tunings were repeated 200 times by randomly selecting 75% of a train set samples. The prediction performance was estimated using the Area Under the receiver operation characteristics Curve (AUC).

### DETAILED DESCRIPTION OF THE INVENTION

Autism Spectrum Disorder (ASD) is a common pediatric cognitive disorder with high heritability although no single gene or locus has been identified to date that explains a majority of cases diagnosed. Earlier diagnosis and behavioral intervention changes the outcome⁴, thus to distinguish the patients with ASD based on a molecular signature from unaffected children would be of great utility in diagnosis and in underpinning the genetic and molecular basis of ASD. No single causative gene or chromosomal locus, however, has been identified to date that explains a majority of cases diagnosed. Current consensus is that the inherited component of ASD is a result of mutations in multiple genes associated with the etiopathology of this heterogeneous developmental condition. Not surprisingly then, the rubric of idiopathic autism is only very gradually shrinking with the discovery of mutations in genes that individually account for 1% or less of all cases in any given ASD cohort (e.g., SHANKS, NLGN3, and NLGN4X)(reviewed in ref.²) and total no more than 2-4%. Copy-number variations appear to account for up to another 10% of genetic contributions to ASD⁵. These multiple mutations can result in a small number of characteristic gene expression signatures in two ways: ASD may result from gene interactions, in which case its essential signature may reflect changes in expression of many genes or ASD may be a constellation of single gene disorders. Present evidence suggests that many of these single gene disorders converge on common mechanisms, so that even for multiple, single gene disorders, there may be a convergent signature in gene expression.

Studies of expression in the brain have been limited to postmortem samples⁶⁻⁸ and these have been notable for gene and protein expression of immune-related pathways (e.g., TNFα, IL1R, and NF-κB systems) in ASD. Numerous lines of evidence suggest that measurements in tissue that are not primarily involved in a disease can also reveal disease signatures and several investigators have demonstrated differential expression of genes in peripheral white blood cells in disorders of the central nervous system⁹⁻¹². To this point, Sullivan et al.¹³ have established a shared expression profile between different CNS tissues and the blood suggesting the use of peripheral blood expression as a surrogate for the brain. Moreover individual gene expression variations of multiple brain regions were correlated well with those of blood in non-human primate¹⁴. Recently, gene expression profiles of lymphoblastoid cell lines were shown to distinguish between different forms of ASD caused by defined genetic lesions (Fragile X syndrome and chromosome 15q duplication) and normal controls¹⁵, and small studies of patients phenotypically defined with ASD have shown differential expression of genes in their peripheral blood cells¹⁶ and in the function of T cell subsets¹⁷. These results are mirrored by proteomic studies of serum, which suggest systematic differences between patients with ASD and controls¹⁸. Thus fresh peripheral blood cells might serve as diagnostic and prognostic surrogate for gene expression in the developing nervous system.

Applicants disclose herein methods that accurately classify patients diagnosed with ASD using gene expression patterns (profiles). Gene expression profiles were obtained from 196 patients with ASDs and 182 controls enrolled in Boston area hospitals. A 330-gene expression signature (ASD330) was developed on one sample cohort (P1) using a machine-learning algorithm, and tested the performance with independently collected second population (P2). Next the gene expression profiles from postmortem brain samples of 11 patients with ASD and 11 controls were prepared to test the possibility of using the blood gene expression signature as a surrogate.

Disclosed herein are the results of a profiling study with peripheral blood gene expression data from 196 patients with ASD and 182 controls enrolled in Boston area hospitals. Applicants developed an expression signature containing 330 genes that achieves 88% cross-validation accuracy on one sample cohort of 97 ASDs and 73 controls. Moreover, this model achieves 78% in an independent population of 99 ASDs and 109 controls. Certain dominant molecular themes for 330 genes used for classification are noteworthy for their association with long-term potentiation and inflammatory pathways heterogeneously distributed across the subjects. This signature also distinguishes postmortem brain gene expression profiles of 11 ASDs from 11 controls.

Methods for characterizing and diagnosing autism spectrum disorder are disclosed herein. The term “autism spectrum disorder” (which may also be referred to herein by the acronym, “ASD”) refers to a spectrum of psychological conditions that cause severe and pervasive impairment in thinking, feeling, language, and the ability to relate to others. Autism spectrum disorder is usually first diagnosed in early childhood and may range in severity from a severe form, called autistic disorder, or autism, through pervasive development disorder not otherwise specified (PDD-NOS), to a much milder form, Asperger syndrome. Autism spectrum disorder may also include two rare disorders, Rett syndrome and childhood disintegrative disorder. As used herein, the phrase “diagnosing autism spectrum disorder” refers to diagnosing, or aiding in diagnosing, an individual as having autism spectrum disorder.

As described herein, a variety of genes are differentially expressed in individuals having autism spectrum disorder compared with individuals not having autism spectrum disorder. An “autism spectrum disorder-associated gene” is a gene whose expression levels are associated with autism spectrum disorder. Examples of autism spectrum disorder-associated genes include, but are not limited to, the genes listed in Table 7. In some embodiments, the autism spectrum disorder associated gene is a gene of Table 4, Table 5, Table 6 or Table 10. As used herein, the term “autism spectrum disorder-associated cell” refers to a cell that expresses one or more autism spectrum disorder-associated genes. In some embodiments, an autism spectrum disorder-associated cell expresses at least two autism spectrum disorder associated genes. As used herein, the term “autism spectrum disorder-associated tissue” is a tissue comprising an autism spectrum disorder-associated cell.

The term “individual”, as used herein, refers to any subject, including, but not limited to, humans and non-human mammals, such as primates, rodents, and dogs. Typically, an individual is a human subject. A human subject may of any appropriate age for the methods disclosed herein. For example, methods disclosed herein may be used to characterize the autism spectrum disorder status of a child, e.g., a human in a range of about 1 to about 12 years old. An individual may be a non-human subject that serves as an animal model of autism spectrum disorder.

Methods are provided herein for characterizing the autism spectrum disorder status of an individual in need thereof. An individual in need of a characterization of autism spectrum disorder status is any individual at risk of, or suspected of, having autism spectrum disorder. An individual's “autism spectrum disorder status” may be characterized as having autism spectrum disorder or as not having autism spectrum disorder.

An individual in need of diagnosis of autism spectrum disorder is any individual at risk of, or suspected of, having autism spectrum disorder. An individual at risk of having autism spectrum disorder may be an individual having one or more risk factors for autism spectrum disorder. Risk factors for autism spectrum disorder include, but are not limited to, a family history of autism spectrum disorder; elevated age of parents; low birth weight; premature birth; presence of a genetic disease associated with autism; and sex (males are more likely to have autism than females). Other risk factors will be apparent to the skilled artisan. An individual suspected of having autism spectrum disorder may be an individual having one or more clinical symptoms of autism spectrum disorder. A variety of clinical symptoms of Autism Spectrum Disorder are known in the art. Examples of such symptoms include, but are not limited to, no babbling by 12 months; no gesturing (pointing, waving goodbye, etc.) by 12 months; no single words by 16 months; no two-word spontaneous phrases (other than instances of echolalia) by 24 months; any loss of any language or social skills, at any age.

The methods disclosed herein may be used in combination with any one of a number of standard diagnostic approaches, including, but not limited to, clinical or psychological observations and/or ASD-related screening modalities, such as, for example, the Modified Checklist for Autism in Toddlers (M-CHAT), the Early Screening of Autistic Traits Questionnaire, and the First Year Inventory to facilitate or aid in the diagnosis of ASD. In some embodiments, methods disclosed herein are used to identify subgroups of ASD.

The methods disclosed herein typically involve determining expression levels of at least one autism spectrum disorder-associated genes in a clinical sample obtained from an individual. The methods may involve determining expression levels of at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, or more autism spectrum disorder-associated genes in a clinical sample obtained from an individual. The methods may involve determining expression levels in a range of 1 to 10, 10 to 20, 20 to 30, 30 to 40, 40 to 50, 50 to 60, 60 to 70, 70 to 80, 80 to 90, 90 to 100, 100 to 200, 200 to 300, or 300 to 400 autism spectrum disorder-associated genes in a clinical sample obtained from an individual.

An expression level determining system may be used in the methods. The term “expression level determining system”, as used herein, refers to a set of components, e.g., equipment, reagents, and methods, e.g., assays, for determining the expression level of a gene in a sample. As will be appreciated by the skilled artisan, the components of an expression level determining system will vary depending on the nature of the method used to determining the expression levels.

The expression level of an autism spectrum disorder-associated gene may be determined as the level of an RNA encoded by the gene, in which case, the expression level determining system will typically comprise components and methods useful for determining levels of nucleic acids. The expression level determining system may comprises, for example, a hybridization-based assay, and related equipment and reagents, for determining the level of the RNA in the clinical sample. Hybridization-based assays are well known in the art and include, but are not limited to, oligonucleotide array assays (e.g., microarray assays), cDNA array assays, oligonucleotide conjugated bead assays (e.g., Multiplex Bead-based Luminex® Assays), molecular inversion probe assay, serial analysis of gene expression (SAGE) assay, RNase Protein Assay, northern blot assay, an in situ hybridization assay, and an RT-PCR assay. Multiplex systems, such as oligonucleotide arrays or bead-based nucleic acid assay systems are particularly useful for evaluating levels of a plurality of nucleic acids in simultaneously. RNA-Seq (mRNA sequencing using Ultra High throughput or Next Generation Sequencing) may also be used to determine expression levels. Other appropriate methods for determining levels of nucleic acids will be apparent to the skilled artisan.

The expression level of an autism spectrum disorder-associated gene may be determined as the level of a protein encoded by the gene, in which case, the expression level determining system will comprise components and methods useful for determining levels of proteins. The expression level determining system may comprises, for example, antibody-based assay, and related equipment and reagents, for determining the level of the protein in the clinical sample. Antibody-based assays are well known in the art and include, but are not limited to, antibody array assays, antibody conjugated-bead assays, enzyme-linked immuno-sorbent (ELISA) assays, immunofluorescence microscopy assays, and immunoblot assays. Other methods for determining protein levels include mass spectroscopy, spectrophotometry, and enzymatic assays. Still other appropriate methods for determining levels of proteins will be apparent to the skilled artisan.

As used herein, a “level” refers to a value indicative of the amount or occurrence of a molecule, e.g., a protein, a nucleic acid, e.g., RNA. A level may be an absolute value, e.g., a quantity of a molecule in a sample, or a relative value, e.g., a quantity of a molecule in a sample relative to the quantity of the molecule in a reference sample (control sample). The level may also be a binary value indicating the presence or absence of a molecule. For example, a molecule may be identified as being present in a sample when a measurement of the quantity of the molecule in the sample, e.g., a fluorescence measurement from a PCR reaction or microarray, exceeds a background value. Similarly, a molecule may be identified as being absent from a sample (or undetectable in the sample) when a measurement of the quantity of the molecule in the sample is at or below background value.

The methods frequently involve obtaining a clinical sample from the individual. As used herein, the phrase “obtaining a clinical sample” refers to any process for directly or indirectly acquiring a clinical sample from an individual. For example, a clinical sample may be obtained (e.g., at a point-of-care facility, e.g., a physician's office, a hospital) by procuring a tissue or fluid sample (e.g., blood draw, spinal tap) from a individual. Alternatively, a clinical sample may be obtained by receiving the clinical sample (e.g., at a laboratory facility) from one or more persons who procured the sample directly from the individual.

The term “clinical sample” refers to a sample derived from an individual, e.g., a patient. Clinical samples include, but are not limited to, tissue, e.g., brain tissue, cerebrospinal fluid, blood, blood fractions such as serum including fetal serum (e.g., SFC) and plasma, blood cells (e.g., white blood cells), sputum, tissue or fine needle biopsy samples, urine, peritoneal fluid, and pleural fluid, or cells there from. A clinical sample comprises a tissue, a cell, and/or a biomolecule, e.g., an RNA, protein. Frequently, the clinical sample is a sample of peripheral blood, brain tissue, or spinal fluid.

It is to be understood that a clinical sample may be processed in any appropriate manner to facilitate determining expression levels of autism spectrum disorder-associated genes. For example, biochemical, mechanical and/or thermal processing methods may be appropriately used to isolate a biomolecule of interest, e.g., RNA, protein, from a clinical sample. A RNA sample may be isolated from a clinical sample by processing the clinical sample using methods well known in the art and levels of an RNA encoded by an autism spectrum disorder-associated gene may be determined in the RNA sample. A protein sample may be isolated from a clinical sample by processing the clinical sample using methods well known in the art and levels of a protein encoded by an autism spectrum disorder-associated gene may be determined in the protein sample. The expression levels of autism spectrum disorder-associated genes may also be determined in a clinical sample directly.

The methods disclosed herein also typically comprise comparing expression levels of autism spectrum disorder-associated genes with an appropriate reference level. An “appropriate reference level” is an expression level of a particular autism spectrum disorder gene that is indicative of a known autism spectrum disorder status. An appropriate reference level can be determined or can be pre-existing. An appropriate reference level may be an expression level indicative of autism spectrum disorder. For example, an appropriate reference level may be representative of the expression level of an autism spectrum disorder-associated gene in a reference (control) clinical sample obtained from a individual known to have autism spectrum disorder. When an appropriate reference level is indicative of autism spectrum disorder, a lack of a detectable difference between a expression level determined from an individual in need of characterization or diagnosis of autism spectrum disorder and the appropriate reference level may be indicative of autism spectrum disorder in the individual. Alternatively, when an appropriate reference level is indicative of autism spectrum disorder, a difference between an expression level determined from an individual in need of characterization or diagnosis of autism spectrum disorder and the appropriate reference level may be indicative of the individual being free of autism spectrum disorder.

Alternatively, an appropriate reference level may be an expression level indicative of an individual being free of autism spectrum disorder. For example, an appropriate reference level may be representative of the expression level of a particular autism spectrum disorder-associated gene in a reference (control) clinical sample obtained from a individual known to be free of autism spectrum disorder. When an appropriate reference level is indicative of an individual being free of autism spectrum disorder, a difference between an expression level determined from an individual in need of diagnosis of autism spectrum disorder and the appropriate reference level may be indicative of autism spectrum disorder in the individual. Alternatively, when an appropriate reference level is indicative of the individual being free of autism spectrum disorder, a lack of a detectable difference between an expression level determined from an individual in need of diagnosis of autism spectrum disorder and the appropriate reference level may be indicative of the individual being free of autism spectrum disorder.

For example, when a higher level, relative to an appropriate reference level that is indicative of an individual being free of autism spectrum disorder, of at least one autism spectrum disorder-associated gene selected from: ARRB2, AVIL, BTBD14A, CD300LF, CXCL1, CYP4F3, FAM101B, FAM13A1OS, HAL, KCNE3, LOC643072, LTB4R, MAN2A2, MSL-1, NBEAL2, NFAM1, NHS, PLA2G7, REM2, SIRPA, SLC45A4, SULF2, and ZNF746 is identified, the individual's autism spectrum disorder status may be characterized as having autism spectrum disorder. When a lower level, relative to an appropriate reference level that is indicative of an individual being free of autism spectrum disorder, of at least one autism spectrum disorder-associated gene selected from: CCDC50, CD180, CPNE5, MYBL2, PNOC, RASSF6, and SPIB is identified, the individual's autism spectrum disorder status may be characterized as having autism spectrum disorder.

The magnitude of difference between a expression level and an appropriate reference level may vary. For example, a significant difference that indicates an autism spectrum disorder status or diagnosis may be detected when the expression level of an autism spectrum disorder-associated gene in a clinical sample is at least 1%, at least 5%, at least 10%, at least 25%, at least 50%, at least 100%, at least 250%, at least 500%, or at least 1000% higher, or lower, than an appropriate reference level of that gene. Similarly, a significant difference may be detected when the expression level of an autism spectrum disorder-associated gene in a clinical sample is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the appropriate reference level of that gene. Significant differences may be identified by using an appropriate statistical test. Tests for statistical significance are well known in the art and are exemplified in Applied Statistics for Engineers and Scientists by Petruccelli, Chen and Nandram 1999 Reprint Ed.

It is to be understood that a plurality of expression levels may be compared with plurality of appropriate reference levels, e.g., on a gene-by-gene basis, as a vector difference, in order to assess the autism spectrum disorder status of the individual. In such cases, Multivariate Tests, e.g., Hotelling's T² test, may be used to evaluate the significance of observed differences. Such multivariate tests are well known in the art and are exemplified in Applied Multivariate Statistical Analysis by Richard Arnold Johnson and Dean W. Wichern Prentice Hall; 4^{th} edition (Jul. 13, 1998).

The methods may also involve comparing a set of expression levels (referred to as an expression pattern) of autism spectrum disorder-associated genes in a clinical sample obtained from an individual with a plurality of sets of reference levels (referred to as reference patterns), each reference pattern being associated with a known autism spectrum disorder status; identifying the reference pattern that most closely resembles the expression pattern; and associating the known autism spectrum disorder status of the reference pattern with the expression pattern, thereby classifying (characterizing) the autism spectrum disorder status of the individual.

The methods may also involve building or constructing a prediction model, which may also be referred to as a classifier or predictor, that can be used to classify the disease status of an individual. As used herein, an “autism spectrum disorder-classifier” is a prediction model that characterizes the autism spectrum disorder status of an individual based on expression levels determined in a clinical sample obtained from the individual. Typically the model is built using samples for which the classification (autism spectrum disorder status) has already been ascertained. Once the model (classifier) is built, it may be applied to expression levels obtained from a clinical sample in order to classify the autism spectrum disorder status of the individual from which the clinical sample was obtained. Thus, the methods may involve applying an autism spectrum disorder-classifier to the expression levels, such that the autism spectrum disorder-classifier characterizes the autism spectrum disorder status of the individual based on the expression levels. The individual may be further diagnosed, e.g., by a health care provider, based on the characterized autism spectrum disorder status.

A variety of prediction models known in the art may be used as an autism spectrum disorder-classifier. For example, an autism spectrum disorder-classifier may comprises an algorithm selected from logistic regression, partial least squares, linear discriminant analysis, quadratic discriminant analysis, neural network, naïve Bayes, C4.5 decision tree, k-nearest neighbor, random forest, and support vector machine.

The autism spectrum disorder-classifier may be trained on a data set comprising expression levels of the plurality of autism spectrum disorder-associated genes in clinical samples obtained from a plurality of individuals identified as having autism spectrum disorder. For example, the autism spectrum disorder-classifier may be trained on a data set comprising expression levels of a plurality of autism spectrum disorder-associated genes in clinical samples obtained from a plurality of individuals identified as having autism spectrum disorder based on DSM-IV-TR criteria. The training set will typically also comprise control individuals identified as not having autism spectrum disorder, e.g., identified as not satisfying the DSM-IV-TR criteria. As will be appreciated by the skilled artisan, the population of individuals of the training data set may have a variety of characteristics by design, e.g., the characteristics of the population may depend on the characteristics of the individuals for whom diagnostic methods that use the classifier may be useful. For example, the interquartile range of ages of a population in the training data set may be from about 2 years old to about 10 years old, about 1 year old to about 20 years old, about 1 year old to about 30 years old. The median age of a population in the training data set may be about 1 year old, 2 years old, 3 years old, 4 years old, 5 years old, 6 years old, 7 years old, 8 years old, 9 years old, 10 years old, 20 years old, 30 years old, 40 years old, or more. The population may consist of all males or may consist of males and females.

A class prediction strength can also be measured to determine the degree of confidence with which the model classifies a clinical sample. The prediction strength conveys the degree of confidence of the classification of the sample and evaluates when a sample cannot be classified. There may be instances in which a sample is tested, but does not belong, or cannot be reliable assign to, a particular class. This is done by utilizing a threshold wherein a sample which scores above or below the determined threshold is not a sample that can be classified (e.g., a “no call”).

Once a model is built, the validity of the model can be tested using methods known in the art. One way to test the validity of the model is by cross-validation of the dataset. To perform cross-validation, one, or a subset, of the samples is eliminated and the model is built, as described above, without the eliminated sample, forming a “cross-validation model.” The eliminated sample is then classified according to the model, as described herein. This process is done with all the samples, or subsets, of the initial dataset and an error rate is determined. The accuracy the model is then assessed. This model classifies samples to be tested with high accuracy for classes that are known, or classes have been previously ascertained. Another way to validate the model is to apply the model to an independent data set, such as a new clinical sample having an unknown autism spectrum disorder status.

As will be appreciated by the skilled artisan, the strength of the model may be assessed by a variety of parameters including, but not limited to, the accuracy, sensitivity and specificity. Methods for computing accuracy, sensitivity and specificity are known in the art and described herein (See, e.g., the Examples). The autism spectrum disorder-classifier may have an accuracy of at least 60%, at least 65%, at least 70%, at least 75%, at least 80%, at least 85%, at least 90%, at least 95%, at least 99%, or more. The autism spectrum disorder-classifier may have an accuracy in a range of about 60% to 70%, 70% to 80%, 80% to 90%, or 90% to 100%. The autism spectrum disorder-classifier may have an sensitivity of at least 60%, at least 65%, at least 70%, at least 75%, at least 80%, at least 85%, at least 90%, at least 95%, at least 99%, or more. The autism spectrum disorder-classifier may have an sensitivity in a range of about 60% to 70%, 70% to 80%, 80% to 90%, or 90% to 100%. The autism spectrum disorder-classifier may have an specificity of at least 60%, at least 65%, at least 70%, at least 75%, at least 80%, at least 85%, at least 90%, at least 95%, at least 99%, or more. The autism spectrum disorder-classifier may have an specificity in a range of about 60% to 70%, 70% to 80%, 80% to 90%, or 90% to 100%.

Described herein are oligonucleotide (nucleic acid) arrays that are useful in the methods for determining levels of multiple nucleic acids simultaneously. Such arrays may be obtained or produced from commercial sources. Methods for producing nucleic acid arrays are well known in the art. For example, nucleic acid arrays may be constructed by immobilizing to a solid support large numbers of oligonucleotides, polynucleotides, or cDNAs capable of hybridizing to nucleic acids corresponding to mRNAs, or portions thereof. The skilled artisan is also referred to Chapter 22 “Nucleic Acid Arrays” of Current Protocols In Molecular Biology (Eds. Ausubel et al. John Wiley and #38; Sons NY, 2000), International Publication WO00/58516, U.S. Pat. No. 5,677,195 and U.S. Pat. No. 5,445,934 which provide non-limiting examples of methods relating to nucleic acid array construction and use in detection of nucleic acids of interest. In some embodiments, the nucleic acid arrays comprise, or consist essentially of, binding probes for mRNAs of at least 2, at least 5, at least 10, at least 20, at least 50, at least 100, at least 200, at least 300, or more genes selected from Table 7. Kits comprising the oligonucleotide arrays are also provided. Kits may include nucleic acid labeling reagents and instructions for determining expression levels using the arrays.

### EXAMPLES

### Introduction to the Examples

Prior studies^{15-17,24,25} have found differential expression of genes in brain and blood samples. The examples disclosed herein demonstrate patients with ASD may be distinguished from “normal” controls with accuracies of greater than 80% across a population and greater than 67% in a second validation population (and 78% accuracy when going from P2 to P1). The odds ratios entailed by this classification are also high (Table 8). The robustness of this classification across these populations is remarkable, particularly as the two groups were heterogeneous and relatively small. With wishing to be bound by theory, the results suggest that the predictors are either capturing a multiplicity of effects from an equally large number of etiologies or encompassing a smaller number of pathophysiologies that constitute a partially shared end point in ASD in a much larger set of etiologies. This contrasts with the small percentage of ASD cases characterized through genetic mutations to date. The classifying performance of the ASD330 is also intriguing in that it is based on measurements in peripheral blood mononuclear cells (PBMC)'s rather than tissues of the central nervous system. Moreover, these PBMC-borne measures are congruent with those of cerebellar expression and can also be used to accurately classify those brain samples. This congruence is echoed in the concordance of genes with decreased methylation in a separate study to genes with increased expression in this study. The pathways that were found to be enriched include those that are classically thought of as neurodevelopmental (e.g. the Notch signaling pathways)²⁰, and genes in involved long-term potentiation²⁶ and including several genes in the calmodulin pathways such as CREBBP (p-value <0.0001, q-value 0.0028 in P1; p-value 0.14, q-value 0.19 in P2) and MEF2C (p-value 0.0054, q-value 0.016 in P1; p-value <0.0001, q-value <0.0001 in P2). Among the latter group was CREBBP which has been implicated in Rubinstein-Taybi syndrome (mental retardation—sometimes with autistic features—and skeletal abnormalities) and was recently implicated in a candidate gene study of autism although the finding was not replicated in a second population²⁷. The MEF2 target genes such as PCDH10 and C3orf58 (also known as deleted in autism1 (DIA1)) have been implicated in ASD^{28,29}, and PCDH10 was up-regulated in P1 (p-value <0.0001, q-value 0.016). Again, without wishing to be bound by theory, the fact that these expression perturbations were found in PBMC's suggests that there are broad transcriptional changes across multiple tissues in ASD even if the pathology is only apparent in the CNS. The pathways related to the brain neural/synaptic activities identified by others (e.g. Purcell and colleagues) that include GABA and Glutamate receptors such as GABRA5 and AMPA and NMDA receptors, and Reelin (Rein) to be specifically and highly expressed in multiple parts of brain such as cortex, hippocampus, and striatum, were not found significantly differentially regulated here except for RAF1. Dominant themes across all the data sets are those of immune signaling, including the B-cell and natural killer T cell signaling pathways. Although immunological and/or “inflammatory” pathways have only recently been implicated in neurodevelopment³⁰⁻³², there is evidence of immunological changes in patients with ASD including in the CNS (e.g. microglial proliferation⁶, up regulation of cytokines and other messengers typically related to inflammation at the level of mRNA and protein expression⁷), as well as in the peripheral blood (e.g. differences in NK cell, TH1 and TH2 subsets, and serum markers^{17,18}). That is, there is overlap in the immunological “themes” differentially expressed in the PBMC's and those reported by others in proteomic and gene expression profiling in the central nervous system. Moreover, the evidence for autoimmune processes in ASD and epidemiological overlap with other autoimmune disorders is growing ³³. Gene sets related to endosomal trafficking were also enriched (Table 1). Among these genes, lysosome-associated membrane protein-2 (LAMP2) mutation has been reported with a rare case of Danon disease with autism³⁴.

Two data sets (P1 and P2) used in this study were obtained at different times and the methods for RNA acquisition in P1 differed in part from those in P2. Also, the control population in P2 different in differed in ethnicity and in the clinics from which they were drawn. This heterogeneity adds noise to the case vs. control comparison and conversely if the analysis utilized more homogeneous data sets, we would have expected improved accuracy. Further, because the numbers of patients were relatively small it was not possible to achieve large enough subsamples of ASD endophenotypes that might have a more homogenous etiology. The data were collected after diagnosis and not as part of a longitudinal study of individuals. The application of these predictors to a prospective cohort would permit further assess their validity as a diagnostic and prognostic tool. The results obtained from groups with ASD were compared to normal controls not to individuals with other neurodevelopmental disorders.

The examples disclosed herein demonstrate that the use of peripheral blood with expression studies offers significant clinical utility for the diagnosis of ASD. The role of the pathways implicating long-term potentiation and immunological mechanisms in the etiology or effect of ASD appear increasingly prominent across multiple tissues.

Summary of Aspects of the Methods

Patients and control samples. Total of 378 blood and 22 postmortem cerebella samples were collected and interrogated using oligonucleotides microarrays. Affymetrix HG-U133 plus 2 (97 ASDs and 73 controls) and Gene 1.0 ST arrays (99 ASDs and 109 controls) were used for two sets of blood samples, and Exon 1.0 ST arrays (11 ASDs and 11 controls) were used for the brain samples. Microarray data with sample characteristics are available at the Gene Expression Omnibus database (GSE18123).

Prediction Analysis.

Gene expression profiles were subject to a machine-learning method for distinguishing ASD from controls. Two independently collected datasets served as a training set (P1) and a validation set (P2). Informative genes were selected using a cross validation method from the training set (P1) to build the prediction model. The prediction model was tested for classification accuracy with the validation set (P2) and 22 postmortem brain samples. Partial least squares and logistic regression methods were used to select genes for prediction models. See Full Methods for detailed description of procedures.

### Example 1

### Gene Expression Profiles of Peripheral Blood in the Patients with ASD

Patients with ASD were recruited from the Developmental Medicine Center (DMC), the Division of Genetics, and the Department of Neurology at the Children's Hospital Boston (CHB) with additional samples obtained from Boston Medical Center (BMC), Cambridge Health Alliance, Tufts Medical Center, and Mass General Hospital (MGH) in collaboration with the Autism Consortium of Boston. Patients recruited for this study have undergone diagnostic assessment, using the Autism Diagnostic Observation Schedule (ADOS) and the Autism-Diagnostic Interview-Revised (ADI-R), as well as comprehensive clinical genetic testing. Inclusion criteria comprised a diagnosis of ASD by DSM-IV-TR criteria, positive ADOS and ADI-R, and an age >24 months (see Methods). Collection of control samples was performed through partnerships with both the Department of Endocrinology (12 individuals from the P1 group) and Children's Hospital Primary Care Center (CHPCC) (61 individuals from P1 and all 109 from P2). Patients seen in the Endocrine department were identified as healthy children with idiopathic short stature, including genetic short stature and constitutional delay of growth, and were having clinical blood draws. The clinical blood draw results were examined to confirm they were within normal limits (those that were not were withdrawn from the study). Patients seen in the CHPCC for a well-child visit that involved a routine blood draw (for example to obtain lead levels) were offered enrollment. A diagnosis of a chronic disease, mental retardation, autism spectrum disorder, or neurological disorder acted as exclusion criteria from our control group. Postmortem cerebella samples from 11 patients with ASD and 11 controls were obtained from the Brain and Tissue Bank at the University of Maryland and the Harvard Brain Tissue Resource Center under IRB approval.

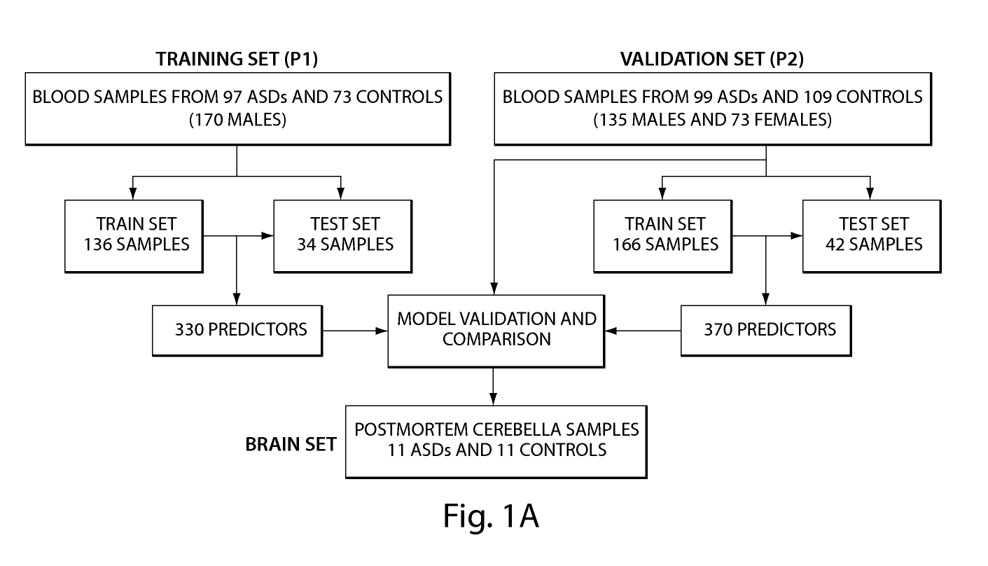

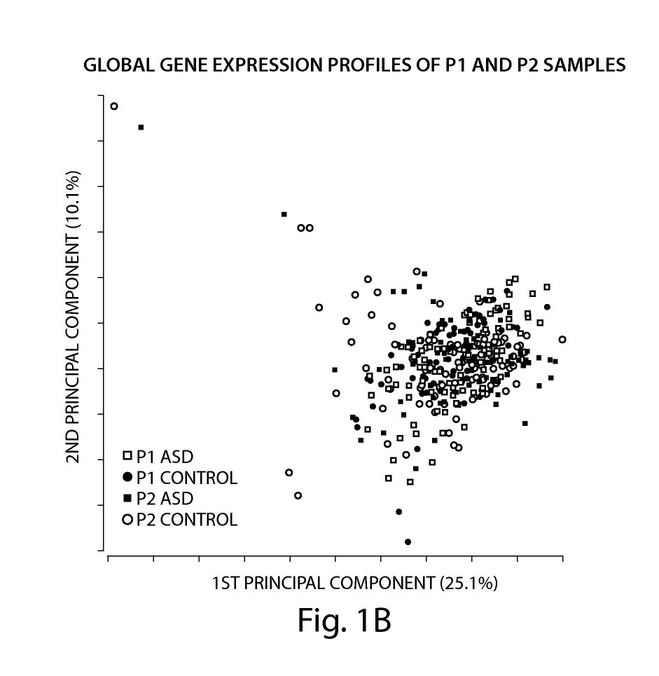

The first sample cohort served as a training set (P1), which encompassed blood gene expression profiles from 97 patients with ASD and 73 controls. Subsequently, 99 patients and 109 controls were recruited for the second sample cohort (P2). To reduce the gender specific gene expression changes that possibly confounds with ASD related gene expression changes, only male samples were recruited in our training set (P1) by study design (FIG. 1a). There was no significant difference in clinical characteristics of ASD between the training and validation set except for gender ratios (p<0.001) (Table 2). Control samples were significantly younger than ASD samples in the training and validation sets (p<0.001), however there was no significant difference between the control samples of the training and validation sets (p=0.98) (Table 3). In the validation set, the proportion of female samples in ASD group (24%) was lower than that of control group (45%) (p=0.002). Independent data sets consisted of 73 (P1) and 109 (P2) control individuals (FIG. 1a). Gene expression profiling of RNA from dataset P1 was conducted using the Human Genome U133 Plus 2.0 microarray platform (Affymetrix, Santa Clara, Calif.) and profiling of RNA from dataset P2 was conducted using the GeneChip Human Gene 1.0 ST Array (Affymetrix, Santa Clara, Calif.). Additionally, 22 brain samples were hybridized to the Affymetrix Exon 1.0 ST microarrays. Microarray data with sample characteristics are available at the Gene Expression Omnibus database (GSE18123). A subset of the gene expression data in 55 ASD and 61 control samples from P1 and 20 ASD and 20 control samples from P2 was further validated using nanoliter reactions and the Universal Probe Library system (Roche, Indianapolis, Ind.) on the Biomark real time PCR system (Fluidigm, South San Francisco, Calif.).

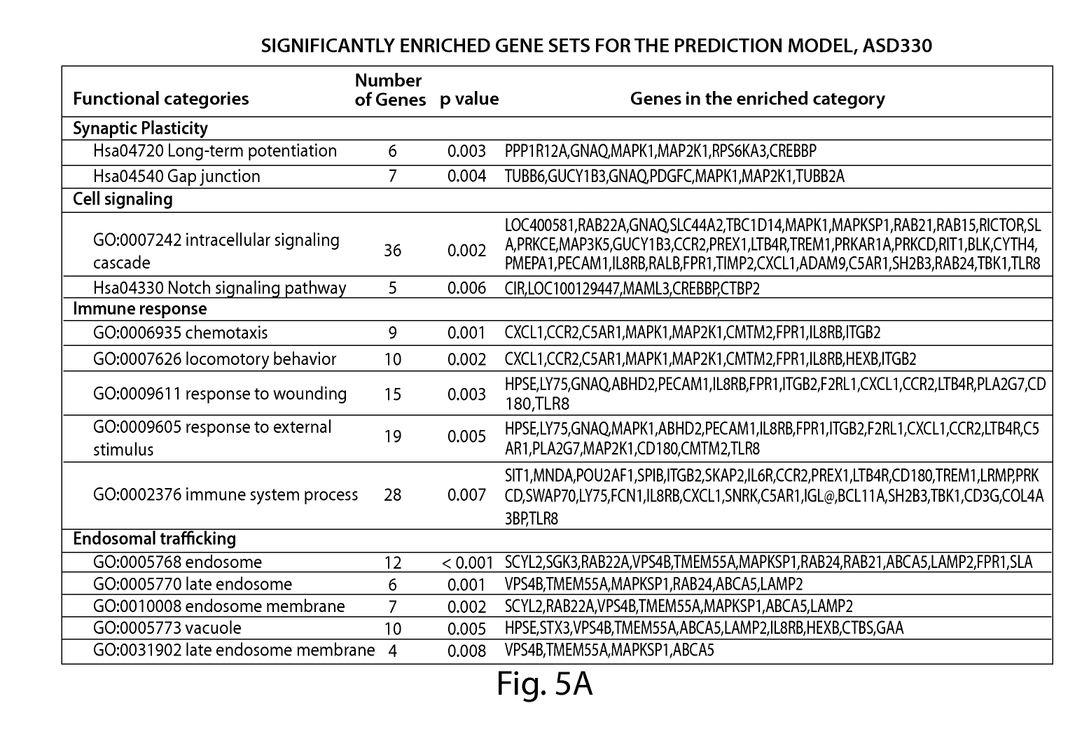

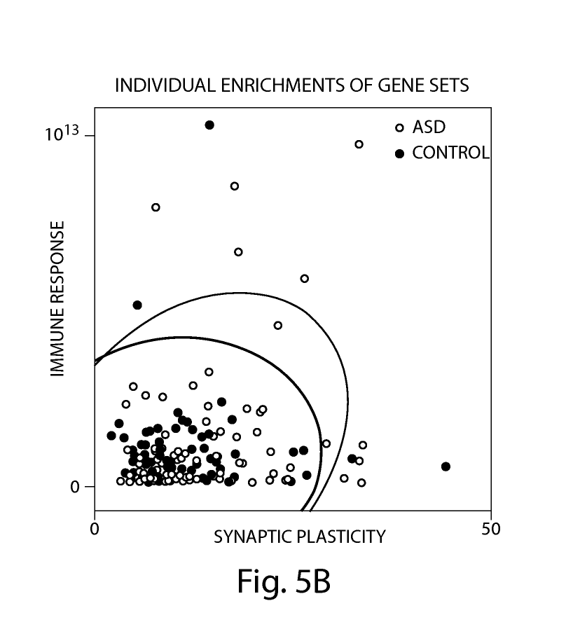

Although the two data sets differ from one another in the time they were acquired, in gender ratios, and in the RNA extraction method, global gene expression profiles of 378 samples did not segregate appreciably by the training and validation sets, or by diagnosis (FIG. 1b). The 4417 topmost differentially expressed genes (false discovery rate, q-value <0.05) are shown in Table 4. Validated 25 of the 27 up-regulated probes (one probe failure, one discordant result) and 7 of the most significant down-regulated genes in probes in (55 ASD and 61 control samples from) P1 samples were further validated using qRT-PCR. Of those 32 qRT-PCR validated genes, 28 were confirmed for differential expressions (Welch's t-test p-value <0.05), and 31 were also differentially expressed in P2 (20 ASD and 20 control samples), see Table 5. Tables 4 and 6 list the genes most differentially expressed in P1 and P2 (q-value <0.05). Common pathways were identified between the P1 and P2 blood data sets (FIG. 5), such as three Gene Ontology categories related to neuron differentiation.

### Example 2

### Blood Gene Expression Signature Accurately Distinguishes the Patients with ASD

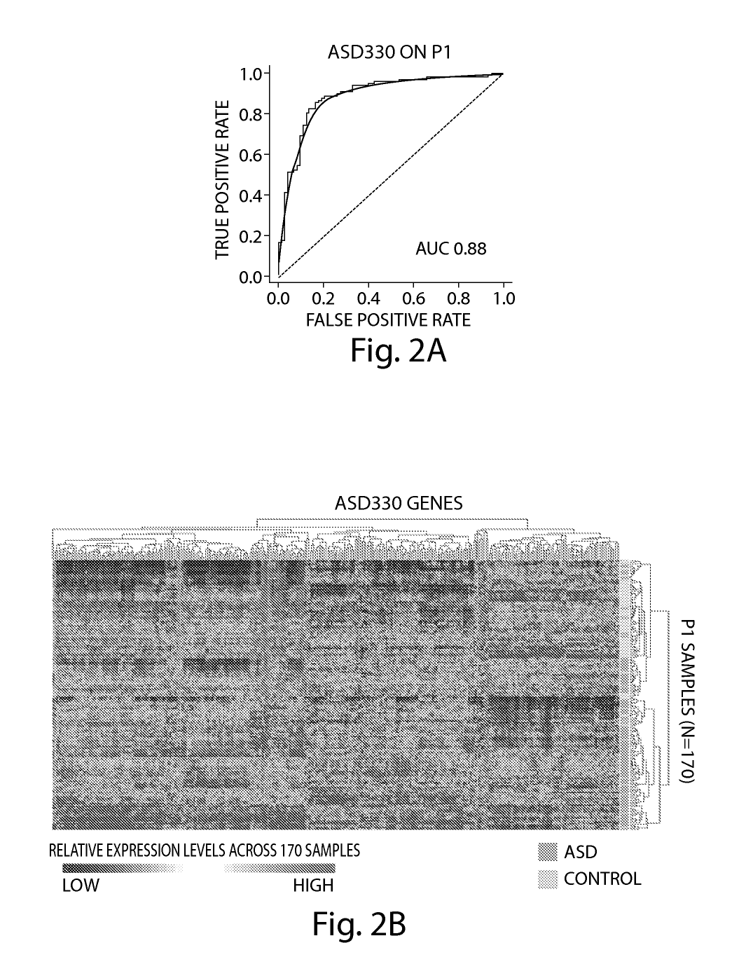

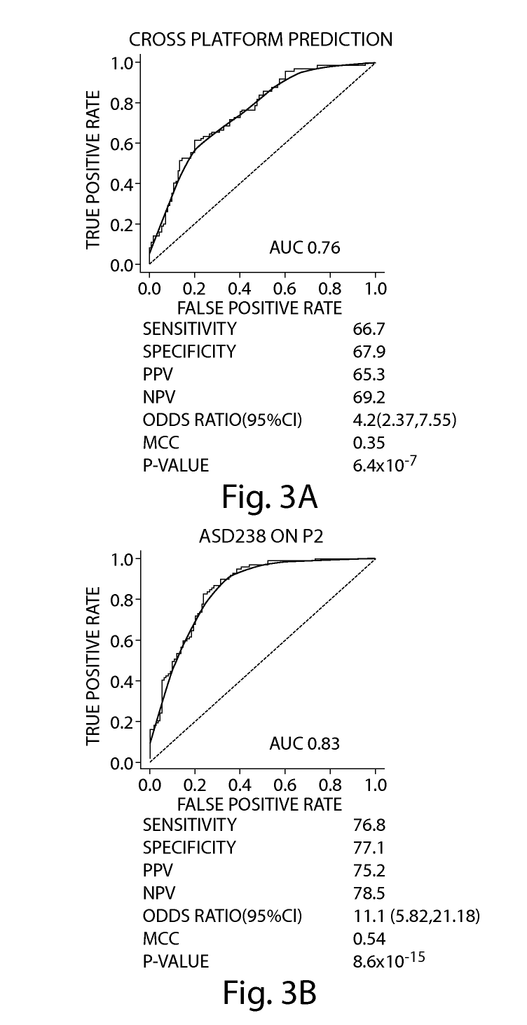

A leave-group out cross-validation strategy was used (see Methods) on P1 to determine the number of genes for the best performing prediction model. The highest accuracy was achieved when top 330 probesets ranked by the partial Area Under the receiver operating characteristic (ROC) Curve (pAUC) scores were used to build the prediction model using a logistic regression or a partial least squares methods. These 330 probesets are designated as ASD330 hereafter (330 probesets are listed in Table 7). The ROC curve for P1 using logistic regression showed overall performance of ASD330 classifier (FIG. 2a, AUC 0.88). The performance of partial least squares or logistic regression was comparable to those of other prediction algorithms (see Methods), thus the classification performance was not attributable to a specific method. Overall average accuracy was 83.1% (range, 75.3% to 88.8%) with 85.9% (range, 70.1 to 93.8%) sensitivity and 79.5% (range, 65.8% to 86.3%) specificity. The average odds ratio was 32.4 (range, 10.8 to 61.6), and the average Matthews correlation coefficient (MCC) was 0.66 (p value <0.001). Misclassified samples could also be identified from the heatmap of the two-way clustering results with ASD330 probesets and the training set samples for not all ASD samples were clustered together (FIG. 2b). When the 330 probe sets from the microarray used in P1 (Affymetrix HG-U133 Plus 2.0) were mapped onto the probesets of the microarray used in P2 (Affymetrix Human Gene 1.0 ST)(see Methods), 238 genes could be mapped reliably (the ASD238). When the prediction model of ASD238 was trained on P1, the performance of predicting P2 samples dropped to an overall accuracy of 67.3% (PPV 65.3% and NPV 69.2%) with an odds ratio of 4.2 (95% Confidence Intervals (CI) 2.37 to 7.55) (FIG. 3a). However, when they were trained on 80% of the P2 samples to predict the remaining 20% of P2, the ASD238 had a performance of 76.9% accuracy (PPV 75.2%, NPV 78.5%, OR 11.1 with 95% CI 5.82 to 21.18) using logistic regression with 5-CV (FIG. 3b).

Although the P1 population recruitment and assays preceded that of P2, for completeness a classifier was developed in the reciprocal fashion starting with P2. The highest accuracy was achieved with a 370-gene classifier (ASD370) with overall accuracy of 77.3% (range, 67.3% to 82.2%) with AUC 0.85 (range, 0.714 to 0.902) (PPV 76.3% (range, 67.0% to 81.7%), NPV 78.7% (range, 67.5% to 83.5%), OR 13.6 (range, 4.2 to 21.3)). When ASD370 was then applied (without retraining) to P1, the overall accuracy was 78.2% (PPV 80%, NPV 75.7%) with OR 12.47 (95% CIs 5.99 to 25.98), as summarized in Table 8.

### Example 3

### Blood Signature Distinguishes Brain Samples

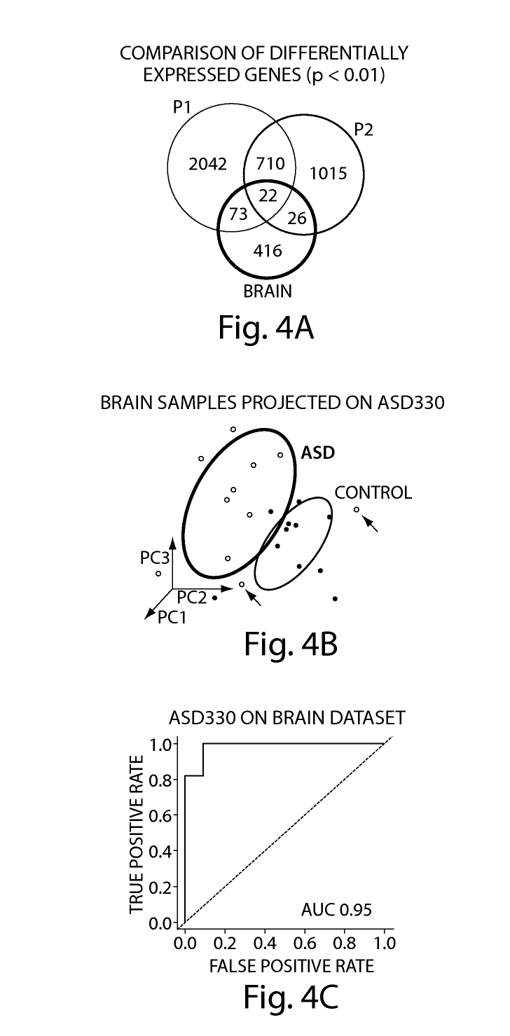

The prediction model from peripheral blood gene expression of our sample cohort was evaluated for its ability to discriminate brain samples from patients with ASD and from controls. 11 postmortem cerebella samples from ASD and 11 samples from controls were obtained from the Brain and Tissue Bank at the University of Maryland and the Harvard Brain Tissue Resource Center under IRB approval, and hybridized to the Affymetrix Human Exon 1.0 ST microarrays. There are 95 common genes between 2847 differentially expressed genes of P1 and 537 genes from brain samples (uncorrected p-value <0.01, Welch's t-test)(FIG. 4a). This overlap was highly significant (p=0.0003) on permutation testing. The ability of the ASD330 classifier to segregate ASD brain samples from control samples was evaluated. The results indicate that the brain samples were significantly segregated from the prediction model developed with the blood profiles (the ASD330 model). There was a clear separation of ASD and control samples (Hotelling's T² test, permutation p-value 0.097)(FIG. 4b)¹⁹. When ASD330 prediction model was used for the brain dataset, 2 samples from postmortem patients with ASD were misclassified as controls (FIG. 4c). These 2 samples (sample ID 7079 and 5666 in Table 9) were identified as outliers when 22 samples were projected to the first three principal components space of ASD330 genes (FIG. 4b, green arrowheads).

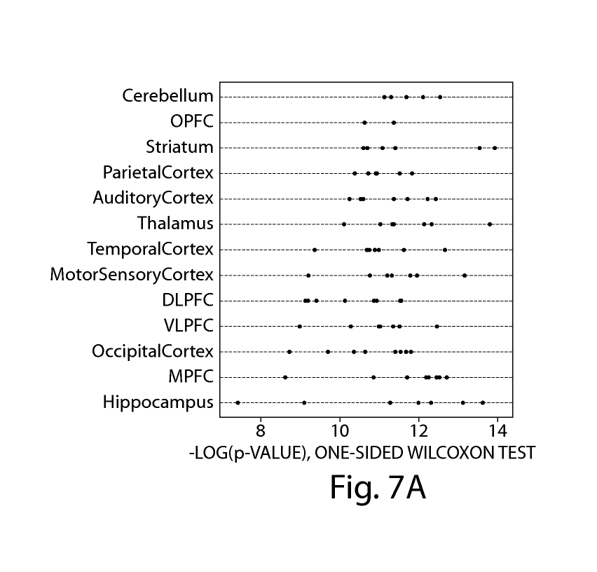

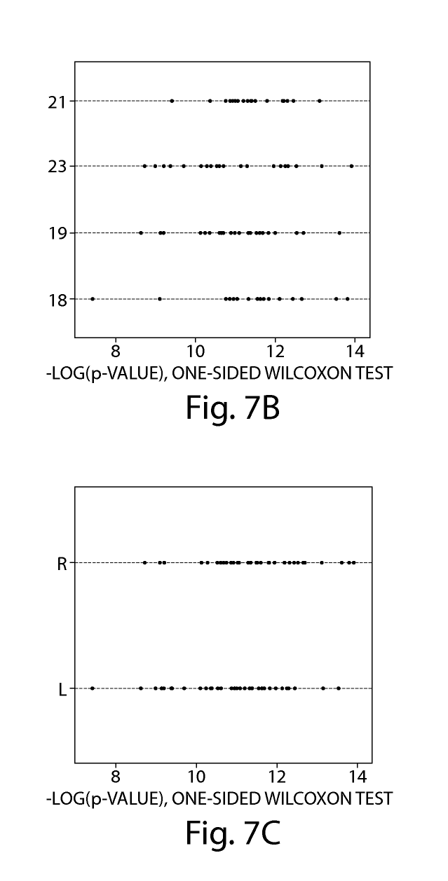

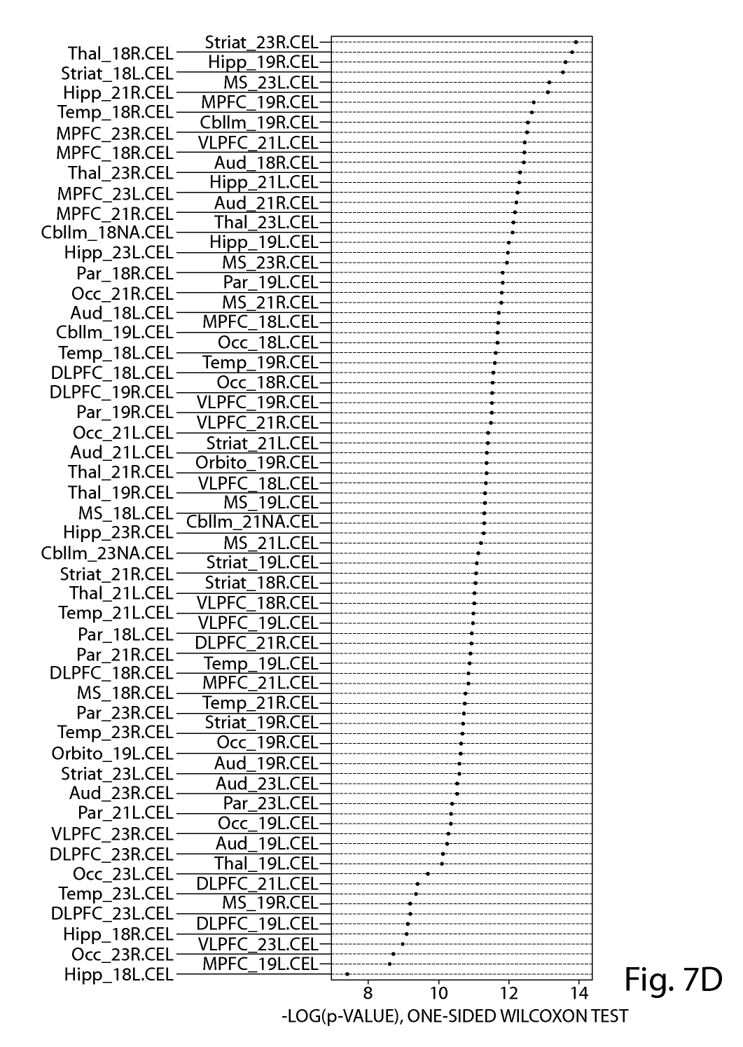

22 genes were differentially expressed in P1, P2, and brain datasets as shown in FIG. 4a. This overlap was deemed to be significant based on a random permutation of the diagnostic labels and calculation of the numbers of overlapping genes by chance (permutation p-value <0.00001). Among these IL18, CD180, NOTCH1²⁰, and TWSG1 are related to immune system process. At least two genes, CCDC6 and TSNAX have been reported in association with ASD. CCDC6 was among the genes in copy number polymorphic region, 10q21.2²¹. Kilpinen et al. reported the allelic diversity of DISC1, DISC2, and TSNAX, clustered on chromosome 1q42, were associated with ASD²². The differentially expressed genes from the postmortem cerebella samples were evaluated to determine if they were exclusively expressed in one part of developing brain or in multiple parts using the fetal human brain transcriptome at different ages (GSE13344)²³. For each brain region, a vector of expression values from differentially expressed genes in ASD (Mann-Whitney-Wilcoxon test uncorrected p-value <0.01) was compared with a vector of expression values obtained from randomly permuted genes. The nonparametric one-sided Wilcoxon rank sum test was used to assess the region enrichments for higher relative expression. Although the brain samples were from the cerebellum, the differential expression does not appear to be correlated to a specific region (FIG. 7a), age (FIG. 7b), or side of the brain (FIG. 7c). Overall cerebellum was the most enriched region, and the other areas such as striatum, thalamus, hippocampus, and medial prefrontal cortex were also enriched (FIG. 7d).

### Example 4

### Biological Themes of Classifier Genes

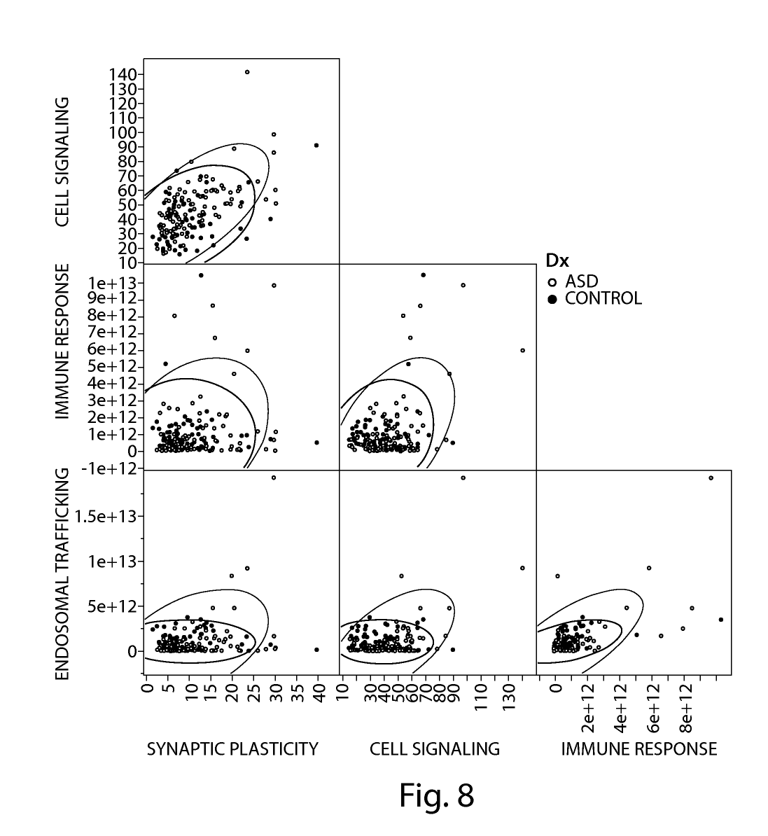

To understand which molecular themes were overrepresented, a gene set enrichment analysis was performed using ASD330 probesets (330 probesets and statistical scores are listed in Table 7). Among the Kyoto Encyclopedia of Genes and Genomes (KEGG) pathways significant pathways included Gap junction (KEGG pathway identifier: hsa04540) and Long-term potentiation (hsa04720) (hypergeometric test p-value 0.0035 and 0.0037 respectively)(Table 1). The long-term potentiation pathway includes NMDA and AMPA glutamate receptors and secondary messenger systems such as calcium and MAPK signaling pathways that converge at cyclic AMP response element-binding protein (CREB) transcriptional pathway. Among the genes of this pathway, CREB binding protein (CREBBP), guanine nucleotide binding protein (G protein), alpha q polypeptide (GNAQ), mitogen activated kinases (MAPK1 and MAP2K1), protein phosphatase 1 subunit (PPP1R12A) and ribosomal protein S6 kinase, polypeptide 3 (RPS6KA3) that interacts with CREBBP were included in ASD330. Moreover 10 genes (CAMK2G, GNAZ, IGF1R, LPAR1, PLA2G4A, PLCB2, PPP1R12A, RAF1, TUBB2A, TUBB6) from Gap junction or Long-term potentiation pathways are differentially expressed in both P1 and P2 (q-value <0.05). In addition, genes involved in the immune system responses and particularly innate immunity were enriched). Chemokine/cytokine related genes (CCR2, CMTM2, CXCL1, IL8RB, and TLR8), receptor for complement C5a (C5AR1(CD88)), second messengers of the chemokine signaling pathway (MAPK1 and MAP2K1), formyl peptide receptor 1(FPR1), and lymphocyte antigens (CD180, ITGB2(CD18), and LY75) recurrently appeared in these Gene Ontology (GO) gene sets, to enriched genesets were clustered into larger categories if significant proportion of genes were found in between GO genesets (Cohen's Kappa >0.5). Among the enriched GO Biological Process (GO-BP) genesets, the immune system process was the most significant (hypergeometric test p-value 0.0007, corresponding q-value 0.013). To determine whether the pathways identified were uniformly or heterogeneously enriched among the subjects, each sample's distance from the multivariate centroid (using T² statistics, see Methods) was calculated for each the functional categories listed in FIG. 5a. Indeed, as exemplified in FIG. 5b, a subgroup of ASD samples was found to be mostly enriched for immune response but not for synaptic plasticity (see FIG. 8 for the other enriched categories).

### Example 5

### Effect of Other Clinical and Demographic Factors on Blood Gene Expression

Potential confounding factors with regard to the ASD330 classifier genes were assess. Among the demographic and clinical features, age at the time of blood drawing and time since last calorie intake were two factors that changed several genes expression. Gene expression level of the Insulin-like growth factor 1 receptor (IGF1R) was marginally correlated with the blood collection time since calorie intake (Spearman's rank correlation coefficient −0.20, p-value 0.0147, N=170). Within the ASD group, the age at the blood collection was correlated with 14 probesets (13 genes) at the significance level of q-value <0.01 (corresponding p<0.00073 using Fisher's r to z transformation of Pearson's correlation coefficients, N=97). These genes are related to transcriptional activities (EBF1, POU2AF1, TCF4, and TOX2), inflammatory response (CD180 and CMTM2), cell growth (NOV), and other functions (CPNE5, CYBASC3, LOC100131043, LOC731484, PMEPA1, and SH3GLP1). The histories of learning, emotional, neurological, autoimmune, and gastrointestinal disorders, and prescribed medication were not significantly correlated with the blood gene expression changes in ASD (N=96, q-value <0.05). 14 probesets were differentially expressed by the history of language disorder (N=96, Positive History N=84, Negative History N=12, q-value <0.05). Interestingly, none of these differentially expressed probesets was found in the ASD330 classifier. 14 probesets, differentially expressed with the history of language disorder, included Chemokine (C-C motif) ligand 23 (CCL23), serine protease 33 (PRSS33), LOC145783, acidic chitinase (CHIA), sphingomyelin phosphodiesterase 3 (SMPD3), the gene encodes Islet-Brain-1 (MAPK81P1), arachidonate 15-lipoxygenase (ALOX15), Tubulin-tyrosine ligase-like protein 9 (TTLL9). The other probesets were not matched with known genes.

Expression profiling may also indicate chromosomal abnormalities, DNA methylation, and epigenetic modifications. For example, an affected male was identified who had high level of X-inactive-specific transcript (XIST) that was comparable to that of females. Subsequent karyotyping of the sample confirmed Klinefelter syndrome, and the case was excluded in this study for further analysis. Epigenetic changes were also reflected in gene expression profiling. Genome wide DNA methylation profiles from 5 patients with ASD and their unaffected siblings were compared to the affected individual's blood gene expression profile. DNA methylation levels were negatively correlated with gene expression (Spearman's rank correlation coefficients, −0.206 to −0.189, p-value <2.2×10⁻¹⁶), and 367 genes were associated with differentially methylated CpG islands (paired t-test uncorrected p-value <0.01). Among these differentially methylated genes, 37 genes were also found by gene expression profiling in the P1 blood data set (Welch's t-tests, q-value <0.05). Moreover, comparison of significant genes from methylation studies of 110 pairs of affected and unaffected siblings, and the differentially expressed genes from P1 dataset revealed 323 unique genes in common (paired t-test controlled for the family effect for DNA methylation data, and Welch's t-test for P1, q-value <0.05 for both datasets). Additionally, ASD330 genes classified 110 pairs of DNA methylation profiles (AUC 0.73 from leave-group out cross validations) independently obtained (personal communication with Dr. Warren at Emory University).

### Example 6

### Materials and Methods

Patients with ASD and Control Samples.

The clinical characteristics of ASD and control samples in the training and validation sets are summarized in Tables 2 and 3. Each proband recruited into the examples disclosed herein underwent an extensive diagnostic evaluation by our trained study staff including the ADI-R, ADOS and cognitive testing. Phenotype information specific to a patient's diagnosis was obtained from these measures. All medical history information obtained on the proband and family members was collected through an interview with the family by a genetic counselor during study enrollment and may include some medical record review. This allows for collection of data regarding co-morbid conditions such as autoimmune disease or neurological disorders including convulsive disorders. In one example, a patient reported by the family to have expressive language disorder would be considered to have a language disorder. However, limited sample size prevents in-depth analysis of endophenotypes and subsets of patients. There was no significant difference in clinical characteristics of ASD between the training and validation set except for gender ratios (p<0.001). Control samples were younger than ASD samples in the training and validation sets (p<0.001), however there was no significant difference between the control samples of the training and validation sets (p=0.98) (Table 3). In the validation set, the proportion of female samples in ASD group (24%) was lower than that of control group (45%) (p=0.002).

Samples and Gene Expression Profiling.

ASD patients were recruited from the Developmental Medicine Center (DMC), the Division of Genetics, and the Department of Neurology at the Children's Hospital Boston (CHB) with additional samples obtained from Boston Medical Center (BMC), Cambridge Health Alliance, Tufts Medical Center, and Mass General Hospital (MGH) in collaboration with the Autism Consortium of Boston. Patients recruited for this study have undergone diagnostic assessment, using the Autism Diagnostic Observation Schedule (ADOS) and the Autism-Diagnostic Interview-Revised (ADI-R), as well as comprehensive clinical genetic testing. Inclusion criteria comprised a diagnosis of ASD by DSM-IV-TR criteria and an age >24 months. Independent data sets consisted of 97 (P1) and 99 (P2) ASD individuals (FIG. 1a). Collection of control samples was performed through partnerships with both the Department of Endocrinology (12 individuals from the P1 group) and Children's Hospital Primary Care Center (CHPCC) (61 individuals from P1 and all 109 from P2). Patients seen in the Endocrine department were identified as healthy children with idiopathic short stature, including genetic short stature and constitutional delay of growth, and were having clinical blood draws. Clinical blood draw results were examined to confirm they were within normal limits (those that were not were withdrawn from the study). Patients seen in the CHPCC for a well-child visit that involves a routine blood draw (for example to obtain lead levels) were offered enrollment. A diagnosis of a chronic disease, mental retardation, autism spectrum disorder, or neurological disorder acts as exclusion criteria from our control group. Independent data sets consisted of 73 (P1) and 109 (P2) control individuals (FIG. 1a). Within the P1 data set, RNA from 42 ASD and 12 control samples was isolated directly from whole blood using the RiboPure Blood Kit (Ambion, Inc, Austin, Tex.). The RiboPure Blood kit is a three step protocol consisting of the lysis of whole blood, RNA purification by phenol/chloroform extraction, and a RNA purification on a glass fiber filter, immediately followed by a DNase treatment. The purified total RNA concentration was assessed using a fluorescent nucleic acid probe from the Quant-iT RiboGreen RNA Assay Kit (Molecular Probes, Carlsbad, Calif.) as measured on a fluorescence-based microplate reader. Quality and quantity of these RNAs were also examined using the Experion System (BioRad, Hercules, Calif.). For the other blood samples, total RNA was extracted from 2.5 ml of whole venous blood using the PAXgene Blood RNA System (PreAnalytix Franklin Lakes, N.J.) according to the manufacturer's instructions for 154 ASD (55 P1, 99 P2) and 170 control samples (61 P1, 109 P2). Samples were kept at room temperature for two hours prior to a centrifugation step that pellets the nucleic acids. A series of washes are completed, followed by a proteinase K digestion step. The cell lysate is homogenized using a shredder column and purified with several washes on a spin column. The samples are DNase I treated and eluted. Quality and quantity of these RNAs were assessed using the Nanodrop spectrophotometer (Thermo Scientific, Waltham, Mass.) and Experion System (BioRad, Herculer, Calif.). For the postmortem brain samples, total RNA was extracted from fresh frozen cerebella samples using the mirVana Isolation kit (Ambion, Inc, Austin, Tex.) according to the manufacturer's instructions. Following mechanical disruption of tissue, the sample is lysed in a denaturing solution, which stabilizes RNA and inactivates RNases. The lysate undergoes Acid-Phenol:Chloroform extraction, which removes most of the cellular components. The sample is further purified over a glass-fiber filter to yield total RNA. Quality and quantity of these RNAs were assessed using the BioAnalyzer system (Agilent, Santa Clara, Calif.).

Gene expression profiling of RNA from dataset P1 was conducted using the Human Genome U133 Plus 2.0 microarray platform (U133p2) (Affymetrix, Santa Clara, Calif.) and profiling of RNA from dataset P2 was conducted using the GeneChip Human Gene 1.0 ST arrays (GeneST). Postmortem brain samples were prepared with Affymetrix Exon 1.0 ST arrays (ExonST). A total of 1 μg RNA (U133p2) or 250 ng (GeneST and ExonST) was processed using established Affymetrix protocols for the generation of biotin-labeled cRNA and the hybridization, staining, and scanning of arrays as outlined in the Affymetrix technical manuals. Briefly, total RNA was converted to double stranded cDNA using an oligo(DT) (U133p2) or T7 primer (GeneST and ExonST). Biotin labeled cRNA was then generated from the cDNA by in vitro transcription. The cRNA was quantified using A260 and fragmented. Fragmented cRNA was hybridized to the appropriate Affymetrix array and scanned on an Affymetrix GeneChip scanner 3000. cRNA from both affected and normal control population groups was prepared in batches of a randomized assortment of the two comparison groups. Microarray data with sample characteristics are available at the Gene Expression Omnibus database (GSE18123).

Real Time Quantitative PCR Validation.

A subset of the gene expression data in 55 ASD and 61 control samples from P1 and 20 ASD and 20 control samples from P2 was further validated using nanoliter reactions and the Universal Probe Library system (Roche Indianapolis, Ind.) on the Biomark real time PCR system (Fluidigm, South San Francisco, Calif.). Following the Biomark protocol, real time quantitative PCR (RT-qPCR) amplifications were carried out in 9 nanoliter reaction volume containing 2× Universal Master Mix (Taqman), hydrolysis Universal Probe library (UPL, Roche), probe-specific primers and preamplified cDNA. Pre-amplification reactions were done in a PTC-200 thermal cycler from MJ Research, per Biomark protocol. Reactions and analysis were performed using a Biomark system (Fluidigm, South San Francisco, Calif.). The cycling program consisted of an initial cycle of 50° C. for 2 minutes and a 10 min incubation at 95° C. followed by 40 cycles of 95° C. for 15 seconds, 70° C. for 5 seconds, and 60° C. for 1 minute. Data was normalized to the housekeeping gene GAPDH, and expressed relative to control samples.

Preprocessing of Microarray Data.

The gene expression levels were calculated using the Probe Log Iterative ERorr (PLIER) algorithm after the normalizing the probe intensities using a quantile method. To match the probeset identifiers from two different platforms used in this study, we used the Best Match subset (affymetrix.com/Auth/support/downloads/comparisons/U133PlusVsHuGene_BestMatch.zip) between two as described in the Affymetrix technical note (affymetrix.com/support/technical/manual/comparison_spreadsheets_manual.pdf). 29,129 out of 54,613 total probesets on U133p2 were best-matched to 17,984 unique probesets of Gene 1.0 ST array, and these matched probesets were used for the cross-platform prediction analysis. The same strategy was used for Affymetrix Exon 1.0 ST array probes. For the genes represented by more than two probesets in U133p2 arrays used for the Training set (P1), genes of which all probesets changed to the same direction were included. Differentially expressed genes of combined three datasets and of each dataset were selected using Welch's t-test and the false discovery rate (FDR, q-value) calculation according to Storey and Tibshirani's³⁵. Multivariate analysis was performed using the Hotelling T² test as previously described in Kong et al¹⁹. Permutation test was used where applicable by randomizing the sample labels to generate background distribution, and the number of permutation was listed. The exact test for categorical data was used. All statistical analysis performed using the R statistical language (http://cran.r-project.org) and prediction analysis was performed using the caret R library package³⁶.

Prediction Analysis.

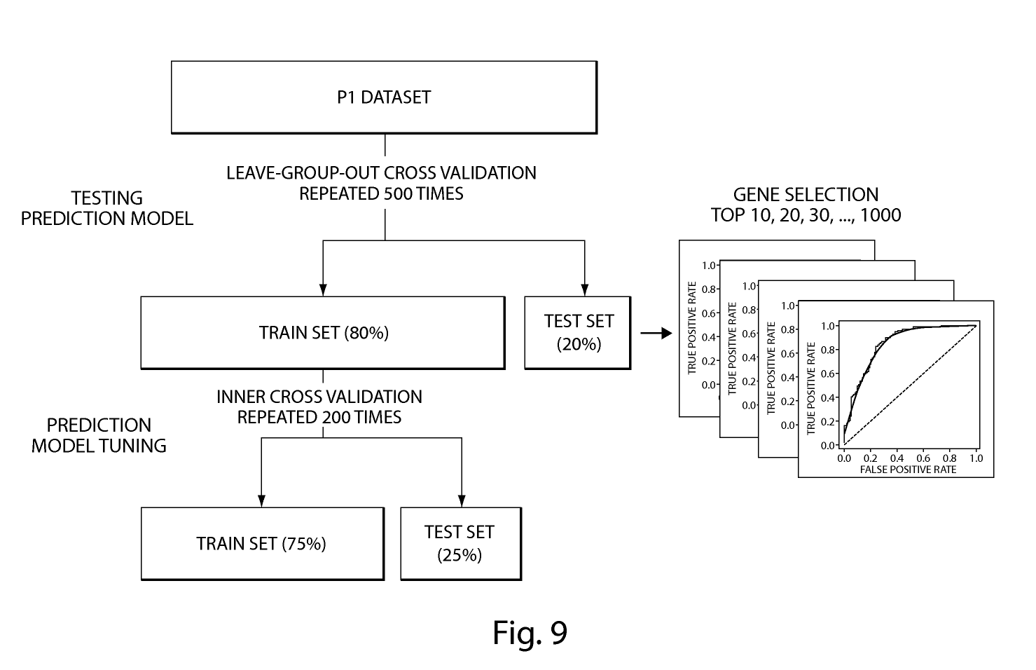

The ability of blood gene expression changes to predict clinical diagnosis was using the logistic regression with five fold cross validations. The prediction analysis was performed in sequential steps; 1) gene selection, 2) setting up a cross-validation strategy in the training set, 3) prediction algorithm selection and build a prediction model, 4) predict the test set, and 5) evaluation of prediction performance (illustrated in FIG. 9). First, all genes (or probesets) were ranked ordered by the partial area under the receiver operation characteristics curve (pAUC) where the partial area was 0.5³⁷. Top N genes (or probesets) varying from 10 to 1000 by 10, and these genes were used to build a prediction model on a test set. A leave-group-out cross validation (LGOCV) strategy was used for the Training Set (P1). In brief, for each top N genes, all P1 samples (N=170) was divided to 80% (a train set) and 20% (a test set), proportion to the number of ASD and controls. This step was repeated 500 times to estimate robust prediction performance. 80% of P1 served as a train set, which a prediction model was tuned. To optimize the prediction model, an inner cross-validation approach was deployed where 75% samples served as an inner train set, and 25% was used as a test set. The inner cross-validation procedure repeated 200 times to find optimal tuning parameters for prediction algorithm. For each top N genes, a total of 10,000 predictions (i.e., 500 LGOCVs×200 inner cross-validations) had been made. The area under the ROC curve (AUC) was used as a primary prediction performance measure to decide the number of genes for final prediction model. For P1 dataset, significant prediction performance was observed when top 330 probesets were used to build prediction models using Partial Least Squares (PLS) or Logistic Regression (LR). The same procedure identified top 370 probsets for P2 dataset. Using a comprehensive machine learning software package, Weka version 3.7.0³⁸, 5 additional prediction methods were tested; Naïve Bayes, C4.5 Decision Tree, k-Nearest Neighbor, Random Forest, and Support Vector Machine using 330 probesets with 5 folds cross validation (5-CV) strategy.

Prediction Performance Measurements.

For each prediction instance, the result are summarized as a 2×2 contingency table with the numbers of True Positive (TP), True Negative (TN), False Positive (FP), and False Negative (FN) predictions. Overall prediction accuracy was calculated as (TP+TN)/N, where N was the total number of samples in a dataset. Sensitivity, specificity, positive predictive value (PPV), and negative predictive value (NPV) were presented as standard measures of prediction performance with the AUC, Matthews Correlation Coefficient (MCC), and Odds Ratio (OR). In a prediction analysis, the output of the prediction methods is continuous probability of being classified as ASD (i.e., threshold), thus there is a trade-off of the amount of false positives among true positives at different threshold. ROC curve summarizes the result at different threshold. AUC was calculated from the ROC curve, i.e., sensitivity (also True Positive Rate (TRP) vs. (1-specificity) (also False Positive Rate (FPR)) as y and x-axis.

For all TP, TN, FP, and FN is related to the performance of any prediction procedure, two metrics were used; MCC and OR that did not discard any of these 4 information. MCC was defined as

$$MCC = \frac{( TP \times TN ) - ( FN \times FP )}{\sqrt{( TP + FP ) ( TP + FN ) ( TN + FP ) ( TN + FN )}} .$$

MCC can range from −1 to 1 where 1 is perfect, 0 is random, and −1 is a total opposite prediction. For the MCC is related to the Chi-square distribution as χ²=N×MCC², p-value was calculated from MCC and average MCC. 95% Confidence Intervals of ln(OR) was calculated as

$$95 \% CI = ln ( OR ) \pm 1.96 \sqrt{( \frac{1}{TP} + \frac{1}{FN} + \frac{1}{FP} + \frac{1}{TN} )}$$

where ln is the natural logarithm.

Functional Enrichment Analysis.

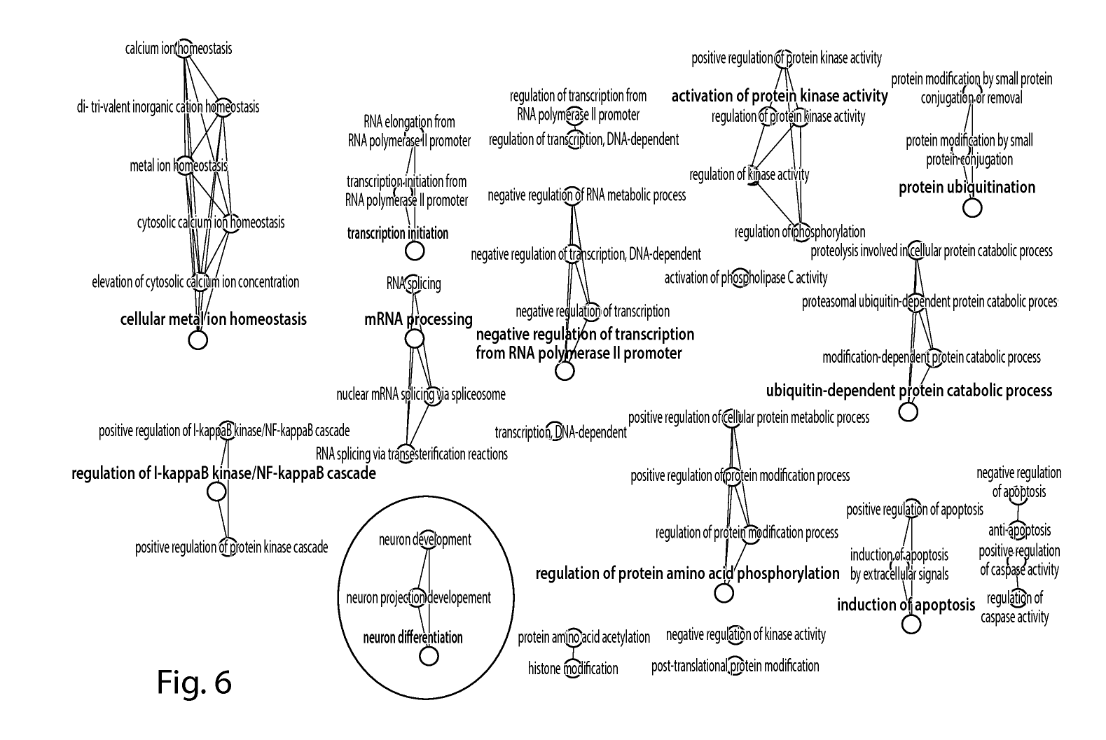

Selected genes for classifiers and the differentially expressed genes were checked for enriched biological theme using the Bioconductor GOstats package³⁹ and DAVID/EASE functional annotation system⁴⁰. Comparative GO analysis was performed at the detailed branch level of GO biological processes using a cytoscape plug-in, the ClueGO⁴¹, and visualized in the Cytoscape⁴² (FIG. 6).

Lengthy table referenced here
US20130123124A1-20130516-T00001
Please refer to the end of the specification for access instructions.

Lengthy table referenced here
US20130123124A1-20130516-T00002
Please refer to the end of the specification for access instructions.

Lengthy table referenced here
US20130123124A1-20130516-T00003
Please refer to the end of the specification for access instructions.

Lengthy table referenced here
US20130123124A1-20130516-T00004
Please refer to the end of the specification for access instructions.

Lengthy table referenced here
US20130123124A1-20130516-T00005
Please refer to the end of the specification for access instructions.

Lengthy table referenced here
US20130123124A1-20130516-T00006
Please refer to the end of the specification for access instructions.

Lengthy table referenced here
US20130123124A1-20130516-T00007
Please refer to the end of the specification for access instructions.

Lengthy table referenced here
US20130123124A1-20130516-T00008
Please refer to the end of the specification for access instructions.

Lengthy table referenced here
US20130123124A1-20130516-T00009
Please refer to the end of the specification for access instructions.

Lengthy table referenced here
US20130123124A1-20130516-T00010
Please refer to the end of the specification for access instructions.

### REFERENCES

1. Prevalence of autism spectrum disorders—autism and developmental disabilities monitoring network, 14 sites, United States, 2002. MMWR Surveill Summ 56, 12-28 (2007).
2. Abrahams, B. S. & Geschwind, D. H. Advances in autism genetics: on the threshold of a new neurobiology. Nat Rev Genet 9, 341-355 (2008).
3. Howlin, P. Autism and diagnostic substitution. Dev Med Child Neurol 50, 325 (2008).
4. Harris, S. L. & Handleman, J. S. Age and IQ at intake as predictors of placement for young children with autism: a four- to six-year follow-up. J Autism Dev Disord 30, 137-142 (2000).
5. Cook, E. H., Jr. & Scherer, S. W. Copy-number variations associated with neuropsychiatric conditions. Nature 455, 919-923 (2008).
6. Vargas, D. L., Nascimbene, C., Krishnan, C., Zimmerman, A. W. & Pardo, C. A. Neuroglial activation and neuroinflammation in the brain of patients with autism. Ann Neurol 57, 67-81 (2005).
7. Garbett, K., et al Immune transcriptome alterations in the temporal cortex of subjects with autism. Neurobiol Dis 30, 303-311 (2008).
8. Fatemi, S. H., Folsom, T. D., Reutiman, T. J. & Lee, S. Expression of astrocytic markers aquaporin 4 and connexin 43 is altered in brains of subjects with autism. Synapse 62, 501-507 (2008).
9. Washizuka, S., Iwamoto, K., Kakiuchi, C., Bundo, M. & Kato, T. Expression of mitochondrial complex I subunit gene NDUFV2 in the lymphoblastoid cells derived from patients with bipolar disorder and schizophrenia. Neurosci Res 63, 199-204 (2009).
10. Padmos, R. C., et al. A discriminating messenger RNA signature for bipolar disorder formed by an aberrant expression of inflammatory genes in monocytes. Arch Gen Psychiatry 65, 395-407 (2008).
11. Coppola, G., et al. Gene expression study on peripheral blood identifies progranulin mutations. Ann Neurol 64, 92-96 (2008).
12. Scherzer, C. R., et al. Molecular markers of early Parkinson's disease based on gene expression in blood. Proc Natl Acad Sci USA 104, 955-960 (2007).
13. Sullivan, P. F., Fan, C. & Perou, C. M. Evaluating the comparability of gene expression in blood and brain. Am J Med Genet B Neuropsychiatr Genet 141B, 261-268 (2006).
14. Jasinska, A. J., et al. Identification of brain transcriptional variation reproduced in peripheral blood: an approach for mapping brain expression traits. Hum Mol Genet 18, 4415-4427 (2009).
15. Nishimura, Y., et al. Genome-wide expression profiling of lymphoblastoid cell lines distinguishes different forms of autism and reveals shared pathways. Hum Mol Genet 16, 1682-1698 (2007).
16. Gregg, J. P., et al. Gene expression changes in children with autism. Genomics 91, 22-29 (2008).
17. Enstrom, A. M., et al. Altered gene expression and function of peripheral blood natural killer cells in children with autism. Brain Behav Immun 23, 124-133 (2009).
18. Corbett, B. A., et al. A proteomic study of serum from children with autism showing differential expression of apolipoproteins and complement proteins. Mol Psychiatry 12, 292-306 (2007).
19. Kong, S. W., Pu, W. T. & Park, P. J. A multivariate approach for integrating genome-wide expression data and biological knowledge. Bioinformatics 22, 2373-2380 (2006).
20. Lasky, J. L. & Wu, H. Notch signaling, brain development, and human disease. Pediatr Res 57, 104R-109R (2005).
21. Sebat, J., et al. Strong association of de novo copy number mutations with autism. Science 316, 445-449 (2007).
22. Kilpinen, H., et al. Association of DISC1 with autism and Asperger syndrome. Mol Psychiatry 13, 187-196 (2008).
23. Johnson, M. B., et al. Functional and evolutionary insights into human brain development through global transcriptome analysis. Neuron 62, 494-509 (2009).
24. Hu, V. W., et al. Gene expression profiling of lymphoblasts from autistic and nonaffected sib pairs: altered pathways in neuronal development and steroid biosynthesis. PLoS One 4, e5775 (2009).
25. Hu, V. W., et al. Gene expression profiling differentiates autism case-controls and phenotypic variants of autism spectrum disorders: evidence for circadian rhythm dysfunction in severe autism. Autism Res 2, 78-97 (2009).
26. Purcell, A. E., Jeon, O. H. & Pevsner, J. The abnormal regulation of gene expression in autistic brain tissue. J Autism Dev Disord 31, 545-549 (2001).
27. Barnby, G., et al. Candidate-gene screening and association analysis at the autism-susceptibility locus on chromosome 16p: evidence of association at GRIN2A and ABAT. Am J Hum Genet 76, 950-966 (2005).
28. Morrow, E. M., et al. Identifying autism loci and genes by tracing recent shared ancestry. Science 321, 218-223 (2008).
29. Flavell, S. W., et al. Genome-wide analysis of MEF2 transcriptional program reveals synaptic target genes and neuronal activity-dependent polyadenylation site selection. Neuron 60, 1022-1038 (2008).
30. Boulanger, L. M. & Shatz, C J Immune signalling in neural development, synaptic plasticity and disease. Nat Rev Neurosci 5, 521-531 (2004).
31. Filipovic, R. & Zecevic, N. The effect of CXCL1 on human fetal oligodendrocyte progenitor cells. Glia 56, 1-15 (2008).
32. Filipovic, R., Jakovcevski, I. & Zecevic, N. GRO-alpha and CXCR2 in the human fetal brain and multiple sclerosis lesions. Dev Neurosci 25, 279-290 (2003).
33. Atladottir, H. O., et al. Association of family history of autoimmune diseases and autism spectrum disorders. Pediatrics 124, 687-694 (2009).
34. Burusnukul, P., de Los Reyes, E. C., Yinger, J. & Boue, D. R. Danon disease: an unusual presentation of autism. Pediatr Neurol 39, 52-54 (2008).
35. Storey, J. D. & Tibshirani, R. Statistical methods for identifying differentially expressed genes in DNA microarrays. Methods Mol Biol 224, 149-157 (2003).
36. Kuhn, M. Building Predictive Models in R Using the caret Package. Journal of Statistical Software 28 (2008).
37. Pepe, M. S., Longton, G., Anderson, G. L. & Schummer, M. Selecting differentially expressed genes from microarray experiments. Biometrics 59, 133-142 (2003).
38. Witten, I. & Frank, E. Data mining: practical machine learning tools and techniques, (Morgan Kaufman, Boston, Mass., 2005).
39. Falcon, S. & Gentleman, R. Using GOstats to test gene lists for GO term association. Bioinformatics 23, 257-258 (2007).
40. Dennis, G., Jr., et al. DAVID: Database for Annotation, Visualization, and Integrated Discovery. Genome Biol 4, P3 (2003).
41. Bindea, G., et al. ClueGO: a Cytoscape plug-in to decipher functionally grouped gene ontology and pathway annotation networks. Bioinformatics 25, 1091-1093 (2009).
42. Cline, M. S., et al. Integration of biological networks and gene expression data using Cytoscape. Nat Protoc 2, 2366-2382 (2007).

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

LENGTHY TABLES
The patent application contains a lengthy table section. A copy of the table is available in electronic form from the USPTO web site (http://seqdata.uspto.gov/?pageRequest=docDetail&DocID=US20130123124A1). An electronic copy of the table will also be available from the USPTO upon request and payment of the fee set forth in 37 CFR 1.19(b)(3).

1. A method of characterizing the autism spectrum disorder status of an individual in need thereof, the method comprising:
(a) obtaining a clinical sample from the individual;
(b) determining expression levels of a plurality of autism spectrum disorder-associated genes in the clinical sample using an expression level determining system,
wherein the autism spectrum disorder-associated genes comprise at least ten genes selected from Table 7; and
(c) comparing each expression level determined in (b) with an appropriate reference level,
wherein the results of comparing in (c) characterize the autism spectrum disorder status of the individual.

2. The method of claim 1, further comprising diagnosing autism spectrum disorder in the individual based on the autism spectrum disorder status.

3. The method of claim 1, wherein the autism spectrum disorder-associated genes comprise at least one of: ARRB2, AVIL, BTBD14A, CCDC50, CD180, CD300LF, CPNE5, CXCL1, CYP4F3, FAMIOIB, FAM13A10S, HAL, KCNE3, LOC643072, LTB4R, MAN2A2, MSL-1, MYBL2, NBEAL2, NFAM1, NHS, PLA2G7, PNOC, RASSF6, REM2, SIRPA, SLC45A4, SPIB, SULF2, TMEM190, ZNF516, and ZNF746.

4. The method of claim 1, wherein a higher level of at least one autism spectrum disorder-associated gene selected from: ARRB2, AVIL, BTBD14A, CD300LF, CXCL1, CYP4F3, FAM101B, FAM13A10S, HAL, KCNE3, LOC643072, LTB4R, MAN2A2, MSL-1, NBEAL2, NFAM1, NHS, PLA2G7, REM2, SIRPA, SLC45A4, SULF2, and ZNF746, compared with an appropriate reference level, characterizes the individual's autism spectrum disorder status as having autism spectrum disorder.

5. The method of claim 1, wherein a lower level of at least one autism spectrum disorder-associated gene selected from: CCDC50, CD180, CPNE5, MYBL2, PNOC, RASSF6, and SPIB, compared with an appropriate reference level, characterizes the individual's autism spectrum disorder status as having autism spectrum disorder.

6. The method of claim 1, wherein the autism spectrum disorder-associated genes comprise at least one of: BCL11A, BLK, C5orf13, CCDC50, CD180, CENPM, CPNE5, CTBP2, EBF1, EIF1AY, FAM105A, FCRL2, HEBP2, IGL@, LOC401233, LRRC6, PLA2G7, PMEPA1, PNN, PNOC, POU2AF1, PRICKLE1, RBP7, SPIB, SULF2, TCF4, TUBB2A, ZNF117, ZNF20, ZNF763, and ZNF830.

7. The method of claim 1, wherein the autism spectrum disorder-associated genes comprise at least one of: TSNAX, SH3BP5L, PPIF, CCDC6, CTSD, IL18, UFM1, MTRF1, LPAR6, TWSG1, MAPKSP1, CD 180, NFYA, TTRAP, ZNF92, CAPZA2, BLK, OSTF1, HSDL2, ATP6V1G1, DCAF12, and NOTCH1.

8. The method of claim 1, wherein the clinical sample is a sample of peripheral blood, brain tissue, or spinal fluid.

9. The method of claim 1, wherein each expression level is a level of an RNA encoded by an autism spectrum disorder-associated gene of the plurality.

10. The method of claim 1, wherein the expression level determining system comprises a hybridization-based assay for determining the level of the RNA in the clinical sample.

11. The method of claim 10, wherein the hybridization-based assay is an oligonucleotide array assay, an oligonucleotide conjugated bead assay, a molecular inversion probe assay, a serial analysis of gene expression (SAGE) assay, or an RT-PCR assay.

12. The method of claim 1, wherein each expression level is a level of a protein encoded by an autism spectrum disorder-associated gene of the plurality.

13. The method of claim 1, wherein the expression level determining system comprises an antibody-based assay for determining the level of the protein in the clinical sample.

14. The method of claim 13, wherein the antibody-based assay is an antibody array assay, an antibody conjugated-bead assay, an enzyme-linked immuno-sorbent (ELISA) assay, or an immunoblot assay.

15. A method of characterizing the autism spectrum disorder status in an individual in need thereof, the method comprising:
(a) obtaining a peripheral blood sample from the individual;
(b) determining expression levels of a plurality of autism spectrum disorder-associated genes in the clinical sample using an expression level determining system,
wherein the autism spectrum disorder-associated genes comprise at least ten genes selected from Table 7; and
(c) applying an autism spectrum disorder-classifier to the expression levels, wherein the autism spectrum disorder-classifier characterizes the autism spectrum disorder status of the individual based on the expression levels.

16. The method of claim 15, further comprising diagnosing autism spectrum disorder in the individual based on the autism spectrum disorder status.

17. The method of claim 15, wherein the autism spectrum disorder-classifier comprises an algorithm selected from logistic regression, partial least squares, linear discriminant analysis, quadratic discriminant analysis, neural network, naive Bayes, C4.5 decision tree, k-nearest neighbor, random forest, and support vector machine.

18. The method of claim 15, wherein the autism spectrum disorder-classifier has an accuracy of at least 75%.

19. The method of claim 15, wherein the autism spectrum disorder-classifier has an accuracy in a range of about 75% to 90%.

20. The method of claim 15, wherein the autism spectrum disorder-classifier has a sensitivity of at least 70%.

21. The method of claim 15, wherein the autism spectrum disorder-classifier has a sensitivity in a range of about 70% to about 95%.

22. The method of claim 15, wherein the autism spectrum disorder-classifier has a specificity of at least 65%.

23. The method of claim 15, wherein the autism spectrum disorder-classifier has a specificity in range of about 65% to about 85%.

24. The method of claim 15, wherein the autism spectrum disorder-classifier is trained on a data set comprising expression levels of the plurality of autism spectrum disorder-associated genes in clinical samples obtained from a plurality of individuals identified as having autism spectrum disorder, wherein the interquartile range of ages of the plurality of individuals identified as having autism spectrum disorder is from about 2 years to about 10 years.

25. The method of claim 15, wherein the autism spectrum disorder-classifier is trained on a data set comprising expression levels of the plurality of autism spectrum disorder-associated genes in clinical samples obtained from a plurality of individuals identified as not having autism spectrum disorder, wherein the interquartile range of ages of the plurality of individuals identified as not having autism spectrum disorder is from about 2 years to about 10 years.

26. The method of claim 15, wherein the autism spectrum disorder-classifier is trained on a data set consisting of expression levels of the plurality of autism spectrum disorder-associated genes in clinical samples obtained from a plurality of male individuals.

27. The method of claim 15, wherein the autism spectrum disorder-classifier is trained on a data set comprising expression levels of the plurality of autism spectrum disorder-associated genes in clinical samples obtained from a plurality of individuals identified as having autism spectrum disorder based on DSM-IV-TR criteria.

28. The method of claim 15, wherein the autism spectrum disorder-associated genes comprise at least one of: BCL11A, BLK, C5orf13, CCDC50, CD180, CENPM, CPNE5, CTBP2, EBF1, EIF1AY, FAM105A, FCRL2, HEBP2, IGL@, LOC401233, LRRC6, PLA2G7, PMEPA1, PNN, PNOC, POU2AF1, PRICKLE1, RBP7, SPIB, SULF2, TCF4, TUBB2A, ZNF117, ZNF20, ZNF763, and ZNF830.

29. The method of claim 15, wherein the autism spectrum disorder-associated genes comprise: TSNAX, SH3BP5L, PPIF, CCDC6, CTSD, HI 8, UFM1, MTRF1, LPAR6, TWSG1, MAPKSP1, CD180, NFYA, TTRAP, ZNF92, CAPZA2, BLK, OSTF1, HSDL2, ATP6V1G1, DCAF12, and NOTCH1.

30. The method of claim 15, wherein the autism spectrum disorder-associated genes comprise at least one of: ARRB2, AVIL, BTBD14A, CCDC50, CD180, CD300LF, CPNE5, CXCL1, CYP4F3, FAM101B, FAM13A10S, HAL, KCNE3, LOC643072, LTB4R, MAN2A2, MSL-1, MYBL2, NBEAL2, NFAM1, NHS, PLA2G7, PNOC, RASSF6, REM2, SIRPA, SLC45A4, SPIB, SULF2, TMEM190, ZNF516, and ZNF746.

31. The method of claim 15, wherein the clinical sample is a sample of peripheral blood, brain tissue, or spinal fluid.

32. The method of claim 15, wherein each expression level is a level of an RNA encoded by an autism spectrum disorder-associated gene of the plurality.

33. The method of claim 15, wherein the expression level determining system comprises a hybridization-based assay for determining the level of the RNA in the clinical sample.

34. The method of claim 33, wherein the hybridization-based assay is an oligonucleotide array assay, an oligonucleotide conjugated bead assay, a molecular inversion probe assay, a serial analysis of gene expression (SAGE) assay, or an RT-PCR assay.

35. The method of claim 15, wherein each expression level is a level of a protein encoded by an autism spectrum disorder-associated gene of the plurality.

36. The method of claim 15, wherein the expression level determining system comprises an antibody-based assay for determining the level of the protein in the clinical sample.

37. The method of claim 36, wherein the antibody-based assay is an antibody array assay, an antibody conjugated-bead assay, an enzyme-linked immuno-sorbent (ELISA) assay, or an immunoblot assay.

38. An array consisting essentially of oligonucleotide probes that hybridize to nucleic acids having sequence correspondence to mRNAs of at least ten autism spectrum disorder-associated genes selected from Table 7.

39. An array consisting essentially of antibodies that bind specifically to proteins encoded by at least ten autism spectrum disorder-associated genes selected from Table 7.

40. A method of monitoring progression of an autism spectrum disorder in an individual in need thereof, the method comprising:
(a) obtaining a clinical sample from the individual;
(b) determining expression levels of a plurality of autism spectrum disorder-associated genes in the clinical sample using an expression level determining system, (c) comparing each expression level determined in (b) with an appropriate reference level, wherein the results of the comparison are indicative of the extent of progression of the autism spectrum disorder in the individual.

41. A method of monitoring progression of an autism spectrum disorder in an individual in need thereof, the method comprising:
(a) obtaining a first clinical sample from the individual,
(b) determining expression levels of a plurality of autism spectrum disorder-associated genes in the first clinical sample using an expression level determining system,
(c) obtaining a second clinical sample from the individual,
(d) determining expression levels of the plurality of autism spectrum disorder-associated genes in the second clinical sample using an expression level determining system,
(e) comparing the expression level of each autism spectrum disorder-associated gene determined in (b) with the expression level determined in (d) of the same autism spectrum disorder associated-gene,
wherein the results of comparing in (e) are indicative of the extent of progression of the autism spectrum disorder in the individual.

42. The method of claim 40, wherein the autism spectrum disorder-associated genes comprise at least ten genes selected from Table 7.

43. A method of monitoring progression of an autism spectrum disorder in an individual in need thereof, the method comprising:
(a) obtaining a first clinical sample from the individual,
(b) obtaining a second clinical sample from the individual,
(c) determining the expression level of an autism spectrum disorder-associated gene in the first clinical sample using an expression level determining system,
(d) determining the expression level of the autism spectrum disorder-associated gene in the second clinical sample using an expression level determining system,
(e) comparing the expression level determined in (c) with the expression level determined in (d),
(f) repeating (c)-(e) for at least one other autism spectrum disorder-associated gene, wherein the results of comparing in (e) for the at least two autism spectrum-associated genes are indicative of the extent of progression of the autism spectrum disorder in the individual.

44. A method of monitoring progression of an autism spectrum disorder in an individual in need thereof, the method comprising:
(a) obtaining a first clinical sample from the individual,
(b) obtaining a second clinical sample from the individual,
(c) determining a first expression pattern comprising expression levels of at least two autism spectrum disorder-associated genes in the first clinical sample using an expression level determining system,
(d) determining a second expression pattern comprising expression levels of at least two autism spectrum disorder-associated genes in the second clinical sample using an expression level determining system,
(e) comparing the first expression pattern with the second expression pattern,
wherein the results of comparing in (e) are indicative of the extent of progression of the autism spectrum disorder in the individual.

45. The method of claim 41, wherein the time between obtaining the first clinical sample and obtaining the second clinical sample is a time sufficient for a change in the severity of the autism spectrum disorder to occur in the individual.

46. The method of claim 41, wherein between obtaining the first clinical sample and obtaining the second clinical sample the individual is treated for the autism spectrum associated disorder.

47. A method of assessing the efficacy of a treatment for an autism spectrum disorder in an individual in need thereof, the method comprising:
(a) obtaining a clinical sample from the individual,
(b) administering a treatment to the individual for the autism spectrum disorder,
(c) determining an expression pattern comprising expression levels of at least two autism spectrum disorder-associated genes in the clinical sample,
(e) comparing the expression pattern with an appropriate reference expression pattern, wherein the appropriate reference expression pattern comprises expression levels of the at least two autism spectrum disorder-associated genes in a clinical sample obtained from an individual who does not have the autism spectrum disorder,
wherein the results of the comparison in (c) are indicative of the efficacy of the treatment.

48. A method of assessing the efficacy of a treatment for an autism spectrum disorder in an individual in need thereof, the method comprising:
(a) obtaining a first clinical sample from the individual,
(b) administering a treatment to the individual for the autism spectrum disorder,
(c) obtaining a second clinical sample from the individual after having administered the treatment to the individual,
(d) determining a first expression pattern comprising expression levels of at least two autism spectrum disorder-associated genes in the first clinical sample,
(e) comparing the first expression pattern with an appropriate reference expression pattern, wherein the appropriate reference expression pattern comprises expression levels of the at least two autism spectrum disorder-associated genes in a clinical sample obtained from an individual who does not have the autism spectrum disorder,
(f) determining a second expression pattern comprising expression levels of at least two autism spectrum disorder-associated genes in the second clinical sample, and
(g) comparing the second expression pattern with the appropriate reference expression pattern, wherein a difference between the second expression pattern and the appropriate reference expression pattern that is less than the difference between the first expression pattern and the appropriate reference pattern is indicative of the treatment being effective.

49. A method for selecting an appropriate dosage of a treatment for an autism spectrum associated disorder in an individual in need thereof, the method comprising:
(i) administering a first dosage of a treatment for an autism spectrum associated disorder to the individual,
(ii) assessing the efficacy of the first dosage of the treatment, in part, by determining at least one expression pattern comprising expression levels of at least two autism spectrum disorder-associated genes in a clinical sample obtained from the individual,
(iii) administering a second dosage of a treatment for an autism spectrum associated disorder in the individual:
(iv) assessing the efficacy of the second dosage of the treatment, in part, by determining at least one expression pattern comprising expression levels of at least two autism spectrum disorder-associated genes in a clinical sample obtained from the individual, wherein the appropriate dosage is selected as the dosage administered in (i) or (iii) that has the greatest efficacy.

50. (canceled)

51. A method for selecting an appropriate dosage of a treatment for an autism spectrum associated disorder in an individual in need thereof, the method comprising:
(i) administering a dosage of a treatment for an autism spectrum associated disorder to the individual;
(ii) assessing the efficacy of the dosage of the treatment, in part, by determining at least one expression pattern comprising expression levels of at least two autism spectrum disorder-associated genes in a clinical sample obtained from the individual, and
(iii) selecting the dosage as being appropriate for the treatment for the autism spectrum associated disorder in the individual, if the efficacy determined in (ii) is at or above a threshold level, wherein the threshold level is an efficacy level at or above which a treatment substantially improves at least one symptom of an autism spectrum disorder.

52. A method for identifying an agent useful for treating an autism spectrum associated disorder in an individual in need thereof, the method comprising:
(i) contacting an autism spectrum associated disorder-cell with a test agent,
(ii) determining at least one expression pattern comprising expression levels of at least two autism spectrum disorder-associated genes in the autism spectrum disorder-associated cell,
(iii) comparing the at least one expression pattern with a test expression pattern, and
(iv) identifying the agent as being useful for treating the autism spectrum associated disorder based on the comparison in (iii).

53. The method of claim 52, wherein test expression pattern is an expression pattern indicative of an individual who does not have the autism spectrum disorder, and wherein a decrease in a difference between the at least one expression pattern and the test expression pattern resulting from contacting the autism spectrum disorder-associated cell with the test agent identifies the test agent as being useful for the treatment of the autism spectrum associated disorder.

54. The method of claim 52, wherein the autism spectrum disorder-associated cell is contacted with the test agent in (i) in vivo.

55. The method of claim 52, wherein the autism spectrum disorder-associated cell is contacted with the test agent in (i) in vitro.
